(19) United States
(12) Patent Application Publication
(10) Pub. No.: US 2021/0206806 A1
(43) Pub. Date: Jul. 8, 2021

(54) PEPTIDIC LINKER WITH REDUCED POST-TRANSLATIONAL MODIFICATION

(71) Applicant: Hoffmann-La Roche Inc., Little Falls, NJ (US)

(72) Inventors: Vincent Larraillet, Penzberg (DE); Michael Molhoj, Munchen (DE); Oksana Tyshchuk, Munchen (DE)

(73) Assignee: Hoffmann-La Roche Inc., Little Falls, NJ (US)

(21) Appl. No.: 17/111,820

(22) Filed: Dec. 4, 2020

Related U.S. Application Data
(63) Continuation of application No. PCT/EP2019/063919, filed on May 29, 2019.

(30) Foreign Application Priority Data
Jun. 8, 2018 (EP) 18176698.1

(51) Int. Cl. C07K 7/08; C07K 16/00

(52) U.S. Cl. CPC C07K 7/08; C07K 16/00; C07K 2317/31; C07K 2317/41; C07K 2319/30

(57) ABSTRACT

Herein is reported a fusion polypeptide comprising the amino acid sequence GnSGmX1X2X3 (SEQ ID NO: 02), wherein X1 can be any amino acid residue except for serine, threonine and proline, wherein X2 and X3 can be independently of each other any amino acid residue, wherein n=1, 2, 3 or 4, and wherein m=3, 4 or 5.

### CROSS REFERENCE TO RELATED APPLICATIONS

This application is a continuation of International Application No. PCT/EP2019/063919, filed May 29, 2019, claiming priority to foreign application number 18176698.1 filed Jun. 8, 2018, which are incorporated herein by reference in their entireties.

### SEQUENCE LISTING

This application contains a Sequence Listing which has been submitted electronically in ASCII format and is hereby incorporated by reference in its entirety. Said ASCII copy, created on Dec. 1, 2020, is named Sequence_listing.txt and is 8,660 bytes in size.

The current invention is in the field of recombinant polypeptide production. In more detail, herein is reported a new peptidic linker that results in less post-translational modification and thereby less product heterogeneity, especially in the recombinant production of fusion polypeptides.

### BACKGROUND OF THE INVENTION

The introduction of multispecific antibodies recombinantly produced in eukaryotic cells resulted in the development of a large number of different molecule formats. In some of these a domain architecture is used wherein different, non-naturally associated domains are fused to each other using a linker. If recombinant production of the fusion polypeptide is intended this linker needs to be an encodeable linker, such as e.g. a peptidic linker.

Peptidic linkers are synthetic amino acid sequences that are employed to connect different polypeptide domains. A peptidic linker generally consists of a linear chain of amino acids wherein the 20 naturally occurring amino acids are the monomeric building blocks which are connected by peptide bonds. The peptidic linker can have a length of from 1 to 50 amino acid residues, with lengths of between 3 and 25 amino acid residues being selected in most cases. A peptidic linker may contain repetitive amino acid sequences. The peptidic linker has the function to ensure that the modules connected by the linker can perform their biological activity by allowing the domains to fold correctly and to be presented properly. Mostly the peptidic linker is a synthetic peptidic linker that is designated to be rich in glycine, glutamine, and/or serine residues. These residues are arranged in small repetitive units of up to five amino acids, such as GGGS or GGGGS (SEQ ID NO: 16 and 17, respectively). The small repetitive unit may be repeated for two to five times to form a multimeric unit, such as e.g. (GGGS)₂ or (GGGGS)₂.

Unfortunately, upon recombinant production of fusion polypeptide comprising peptidic linkers in eukaryotic host cells, amino acids residues within the peptidic linker can serve as substrates for in vivo post-translational modification. The addition of post-translational modification results in an increased heterogeneity of the recombinantly produced fusion polypeptide. Thus, new peptidic linkers that reduce or even eliminate the addition of post-translational modifications would enable the recombinant production of more homogenous fusion polypeptide preparations.

In the publication of Spahr et al. (MAbs 9 (2017) 812-819) a hydroxyproline in a GGGGP (SEQ ID NO: 18) linker was reported.

Shi, Y., reported about biochemical and structural investigations that advance the mechanistic understanding of the three major classes of protein serine/threonine phosphatases, with a focus on PP2A (Cell 139 (2009) 468-484).

Baslé, E., et al. reported about protein chemical modifications on endogenous amino acids (Chem. Biol. 17 (2010) 213-227).

In WO 2003/062276 polypeptide variants were disclosed comprising the ligand binding domains of cytokines which are linked via flexible polypeptide linker molecules.

WO 2011/161260 disclosed anticancer fusion proteins.

WO 2012/088461 disclosed that linker peptides which lack the amino acid sequence GSG reduce or eliminate the addition of posttranslational modifications to the polypeptides which comprise them.

WO 2014/085621 disclosed therapeutic fusion proteins useful to treat lysosomal storage diseases and methods for treating such diseases.

WO 2015/091130 disclosed a method for recombinantly producing a polypeptide in soluble form comprising the steps of transfecting a eukaryotic cell with a nucleic acid encoding the polypeptide, whereby the polypeptide has been modified (compared to the wild-type polypeptide) by the introduction of one or more artificial glycosylation sites, cultivating the transfected cell in a cultivation medium, and recovering the polypeptide from the cultivation medium, whereby the yield (determined after one purification step) of monomeric polypeptide is increased by at least 100% compared to the wild-type polypeptide.

WO 2016/115511 disclosed VEGF variant polypeptide compositions.

WO 2011/161260 disclosed a fusion protein comprising domain (a) which is the functional fragment of soluble hTRAIL protein sequence, and domain (b) which is the sequence of a pro-apoptotic effector peptide.

WO 2016/120216 disclosed a polypeptide encoding a chimeric antigen receptor comprising at least one extracellular binding domain that comprises a scFv formed by at least a VH chain and a VL chain specific to an antigen, wherein said extracellular binding domain comprises at least one mAb-specific epitope.

Spencer, D., et al., disclosed that O-xylosylation in a recombinant protein is directed at a common motif on glycine-serine linkers (J. Pharm. Sci 102 (2013) 3920-3924).

Wen, D., et al., disclosed investigations of O-Xylosylation in engineered proteins containing a (GGGGS)n (SEQ ID NO: 17) linker (Anal. Chem. 85 (2013) 4805-4812).

Peter-Katalinić, J., disclosed that cell surface and extracellular proteins are O-glycosylated, where the most abundant type of O-glycosylation in proteins is the GalNAc attachment to serine or threonine in the protein chain by an a-glycosidic linkage (Meth. Enzymol. 405 (2005) 139-171).

Plomp, R., et al. disclosed hinge-region O-glycosylation of human immunoglobulin G3 (Mol. Cell. Prot. 14 (2015) 1373-1384).

U.S. Pat. No. 9,409,960 discloses linker peptides and polypeptides comprising same, wherein linker peptides which lack the amino acid sequence GSG reduce or eliminate the addition of posttranslational modifications to the polypeptides which comprise them.

### SUMMARY

The invention is based, at least in part, on the finding that glycine-serine peptidic linkers, which lack at least the C-terminal serine residue or which even lack all serine residues resulting in a pure glycine linker, reduce or even eliminate the addition of post-translational modifications to the fusion polypeptides in which these are contained. In order to achieve this, the polypeptide C-terminal to the peptidic linker shall not contain a serine, threonine or proline reside at its N-terminus, i.e. the first amino acid residue after the peptidic linker shall not be a serine, threonine or proline residue.

More specifically, the peptidic linkers as reported herein reduce or even eliminate the ability of enzymes to add post-translational modifications, such as phosphate groups or carbohydrate moieties, to fusion polypeptides comprising such a peptidic linker, e.g., reduce the ability of xylosyltransferase to link xylose to serine residues.

Thus, by including the peptidic linker as reported herein in fusion polypeptides the homogeneity of recombinantly (in eukaryotic cells) produced fusion polypeptide compositions and preparations can be increased.

One aspect of the invention is a fusion polypeptide comprising the amino acid sequence

(SEQ ID NO: 04)
GyX1

wherein X1 can be any amino acid residue except for serine, threonine and proline, and
wherein y is an integer from and including 3 to 25.

In one embodiment the fusion polypeptide comprises the amino acid sequence

(SEQ ID NO: 05)
GyX1X2X3

wherein X1 can be any amino acid residue except for serine, threonine and proline,
wherein X2 and X3 can be independently of each other any amino acid residue, and
wherein y is an integer from and including 3 to 25.

In one embodiment y is an integer from and including 4 to 20.

In one embodiment y is an integer from and including 5 to 15.

One aspect of the invention is a fusion polypeptide comprising the amino acid sequence

(SEQ ID NO: 01)
GnSGmX1

wherein X1 can be any amino acid residue except for serine, threonine and proline,
wherein n=1, 2, 3 or 4, and
wherein m=3, 4 or 5.

In one embodiment of all aspects of the invention the fusion polypeptide comprises the amino acid sequence

(SEQ ID NO: 02)
GnSGmX1X2X3

wherein X1 can be any amino acid residue except for serine, threonine and proline,
wherein X2 and X3 can be independently of each other any amino acid residue,
wherein n=1, 2, 3 or 4, and
wherein m=3, 4 or 5.

In one embodiment of all aspects of the invention the fusion polypeptide comprises at least three domains
wherein each of the three domains is independently of the other two a polypeptide of at least 10 amino acid residues in length, and
wherein the domains are conjugated to each other via peptide bonds.

In one embodiment of all aspects of the invention the C-terminus of the first domain is conjugated to the N-terminus of the second domain via a peptide bond, and the C-terminus of the second domain is conjugated to the N-terminus of the third domain via a peptide bond.

In one embodiment of all aspects of the invention the fusion polypeptide is a recombinant fusion polypeptide.

In one embodiment of all aspects of the invention the fusion polypeptide is produced in a eukaryotic cell.

In one embodiment of all aspects of the invention the fusion polypeptide has no post-translational modification at S or X1 of SEQ ID NO: 01.

In one embodiment of all aspects of the invention the linear fusion polypeptide has no post-translational modification at X1 of SEQ ID NO: 01.

In one embodiment of all aspects of the invention the post-translational modification is phosphorylation (addition of a phosphate group) and/or glycosylation (addition of a carbohydrate moiety). In one embodiment of all aspects of the invention the glycosylation is xylosylation (the carbohydrate moiety is xylose). In one embodiment of all aspects of the invention the glycosylation is glucosylation (the carbohydrate moiety is glucose).

In one embodiment of all aspects of the invention the fusion polypeptide has reduced post-translational modification at residues S and/or X1 compared to a fusion polypeptide comprising

(SEQ ID NO: 06)
GnSGmX1X2X3

wherein X1 is serine, threonine or proline,
wherein X2 and X3 can be independently of each other any amino acid residue,
wherein n=1, 2, 3 or 4, and
wherein m=3, 4 or 5.

In one embodiment of all aspects of the invention n is 3 and m is 3 or 4. In one preferred embodiment of all aspects of the invention n is 3 and m is 4.

In one embodiment of all aspects of the invention n is 4 and m is 4 or 5. In one preferred embodiment of all aspects of the invention n is 4 and m is 5.

In one embodiment of all aspects of the invention the fusion polypeptide comprises the amino acid sequence

(SEQ ID NO: 03)
GpSGnSGmX1X2X3

wherein X1 can be any amino acid residue except for serine, threonine and proline,
wherein X2 and X3 can be independently of each other any amino acid residue,
wherein n=3 or 4,
wherein m=3, 4 or 5, and
wherein p=3 or 4.

In one embodiment of all aspects of the invention the fusion polypeptide further comprises an antibody Fc-region polypeptide/at least one domain is an antibody Fc-region polypeptide.

In one embodiment of all aspects of the invention the first or/and the third domain is an antibody Fc-region polypeptide.

In one embodiment of all aspects of the invention the fusion polypeptide further comprises a VH domain, a VL domain, as scFv, a scFab, a VH-CH1 pair, a VL-CL pair, a VH-CL pair, a VL-CH1 pair, a receptor or extracellular domain thereof, a receptor binding portion of a ligand, an enzyme, a growth factor, an interleukin, a cytokine, or a chemokine.

In one embodiment of all aspects of the invention the fusion polypeptide further comprises at least one domain selected from the group consisting of a VH domain, a VL domain, as scFv, a scFab, a VH-CH1 pair, a VL-CL pair, a VH-CL pair, a VL-CH1 pair, a receptor or extracellular domain thereof, a receptor binding portion of a ligand, an enzyme, a growth factor, an interleukin, a cytokine, and a chemokine.

In one embodiment of all aspects of the invention the fusion polypeptide is monomeric.

In one embodiment of all aspects of the invention the fusion polypeptide is a linear fusion polypeptide.

Another aspect of the invention is a multimeric molecule comprising at least two polypeptides whereof at least one is a fusion polypeptide as reported herein.

In one embodiment of all aspects of the invention the at least two polypeptides are conjugated to each other by one or more disulfide bonds.

In one embodiment of all aspects of the invention the multimeric molecule is an antibody.

In one embodiment of all aspects of the invention the multimeric molecule is a bispecific antibody.

In one embodiment of all aspects of the invention the multimeric molecule further comprises at least one antibody light chain.

In one embodiment of all aspects of the invention the multimeric molecule further comprises at least one antibody heavy chain.

Another aspect of the invention is a pharmaceutical formulation comprising at least one fusion polypeptide as reported herein or a multimeric molecule as reported herein and optionally a pharmaceutically acceptable carrier.

A further aspect of the invention is a nucleic acid molecule encoding a fusion polypeptide as reported herein.

In one embodiment of all aspects of the invention the nucleic acid molecule is in an expression cassette.

In one embodiment of all aspects of the invention the nucleic acid molecule is in a vector.

Also an aspect of the invention is a set of nucleic acid molecules encoding the polypeptides of the multimeric molecule as reported herein.

In one embodiment of all aspects of the invention each of the nucleic acid molecules is in an expression cassette.

In one embodiment of all aspects of the invention each of the nucleic acid molecules is in a vector.

In one embodiment of all aspects of the invention the nucleic acid molecules are on the same vector.

In one embodiment of all aspects of the invention the nucleic acid molecules are on different vectors.

In one embodiment of all aspects of the invention the nucleic acid molecules are on two vectors.

A further aspect as reported herein is a eukaryotic cell comprising the nucleic acid molecule as reported herein or the set of nucleic acid molecules as reported herein.

Another aspect of the invention is a peptidic linker comprising the amino acid sequence

(SEQ ID NO: 04)
GyX1

wherein X1 can be any amino acid residue except for serine, threonine and proline, and
wherein y is an integer from and including 3 to 25.

Another aspect of the invention is a peptidic linker comprising the amino acid sequence

(SEQ ID NO: 05)
GyX1X2X3

wherein X1 can be any amino acid residue except for serine, threonine and proline,
wherein X2 and X3 can be independently of each other any amino acid residue, and
wherein y is an integer from and including 3 to 25.

In one embodiment y is an integer from and including 4 to 20.

In one embodiment y is an integer from and including 5 to 15.

Another aspect of the invention is a peptidic linker comprising the amino acid sequence

(SEQ ID NO: 01)
GnSGmX1

wherein X1 can be any amino acid residue except for serine, threonine and proline,
wherein n=1, 2, 3 or 4, and
wherein m=3, 4 or 5.

Another aspect of the invention is a peptidic linker comprising the amino acid sequence

(SEQ ID NO: 02)
GnSGmX1X2X3

wherein X1 can be any amino acid residue except for serine, threonine and proline,
wherein X2 and X3 can be independently of each other any amino acid residue,
wherein n=1, 2, 3 or 4, and
wherein m=3, 4 or 5.

Another aspect of the invention is a peptidic linker comprising the amino acid sequence

(SEQ ID NO: 03)
GpSGnSGmX1X2X3

wherein X1 can be any amino acid residue except for serine, threonine and proline,
wherein X2 and X3 can be independently of each other any amino acid residue,
wherein n=1, 2, 3 or 4,
wherein m=3, 4 or 5, and
wherein p=3 or 4.

Another aspect of the invention is a method for producing a fusion polypeptide comprising the following steps:
cultivating the eukaryotic cell according to the invention under conditions where the fusion polypeptide is expressed, and
recovering the fusion polypeptide from the eukaryotic cell or the cultivation medium.
thereby producing a fusion polypeptide.

Another aspect of the invention is a method of producing a fusion polypeptide having reduced levels of post-translational modification comprising:
cultivating the host cell according to claim 38 under conditions where the fusion polypeptide is expressed, and
recovering the fusion polypeptide from the eukaryotic cell or the cultivation medium,
thereby producing a fusion polypeptide having reduced levels of post-translational modifications.

A further aspect of the invention is a method of producing a stabilized fusion polypeptide comprising genetically engineering a fusion protein to comprise a peptidic linker of the invention and causing the fusion polypeptide to be expressed by a eukaryotic cell, and thereby producing a stabilized fusion polypeptide.

Also an aspect of the invention is a composition made by the method according to the invention.

A further aspect of the invention is a method of treating a subject that would benefit from treatment with a fusion polypeptide according to the invention or a multimeric molecule according to the invention, comprising administering a composition according to the invention to the subject.

A further aspect of the invention is the use of a composition according to the invention for the treatment of a disease or disorder.

Also an aspect of the invention is the use of a composition according to the invention for the manufacture of a medicament.

An aspect of the invention is further the fusion polypeptide according to the invention or the multimeric molecule according to the invention for use as a medicament.

Also an aspect of the invention is the use of the fusion polypeptide according to the invention or the multimeric molecule according to the invention in the manufacture of a medicament.

A final aspect of the invention is a method of treating an individual in need of a treatment comprising administering to the individual an effective amount of the fusion polypeptide according to the invention or the multimeric molecule according to the invention.

### DETAILED DESCRIPTION OF EMBODIMENTS OF THE INVENTION

The invention is based, at least in part, on the finding that the use of glycine-serine peptidic linkers, which lack the C-terminal serine residue, reduce or even eliminate the addition of post-translational modifications to said peptidic linker, especially when the peptidic linker is comprised in a fusion polypeptide. In order to achieve this, the polypeptide C-terminal to the peptidic linker shall also not contain a serine, threonine or proline reside at its N-terminus.

More specifically, the peptidic linkers reported herein reduce the ability of enzymes to link secondary modifications, such as phosphate groups or carbohydrate moieties, to fusion polypeptides comprising such a peptidic linker, e.g., reduce the ability of xylosyltransferase to link xylose to polypeptides.

Thus, by using a peptidic linker as reported herein in fusion polypeptides the homogeneity of recombinantly (in eukaryotic cells) produced fusion polypeptide compositions and preparations can be increased.

General information regarding the nucleotide sequences of human immunoglobulins light and heavy chains is given in: Kabat, E. A., et al., Sequences of Proteins of Immunological Interest, 5th ed., Public Health Service, National Institutes of Health, Bethesda, Md. (1991). The amino acid positions of all constant regions and domains of the heavy and light chain can be numbered according to the Kabat numbering system described in Kabat, et al., Sequences of Proteins of Immunological Interest, 5th ed., Public Health Service, National Institutes of Health, Bethesda, Md. (1991) and is referred to as “numbering according to Kabat” herein. Specifically, the Kabat numbering system (see pages 647-660) of Kabat, et al., Sequences of Proteins of Immunological Interest, 5th ed., Public Health Service, National Institutes of Health, Bethesda, Md. (1991) is used for the light chain constant domain CL of kappa and lambda isotype, and the Kabat EU index numbering system (see pages 661-723) is used for the constant heavy chain domains (CH1, Hinge, CH2 and CH3, which is herein further clarified by referring to “numbering according to Kabat EU index” in this case).

It must be noted that as used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein.

It is also to be noted that the terms “comprising”, “including”, and “having” can be used interchangeably.

To a person skilled in the art procedures and methods are well known to convert an amino acid sequence, e.g. of a peptidic linker or fusion polypeptide, into a corresponding encoding nucleic acid sequence. Therefore, a nucleic acid is characterized by its nucleic acid sequence consisting of individual nucleotides and likewise by the amino acid sequence of a peptidic linker or fusion polypeptide encoded thereby.

The use of recombinant DNA technology enables the generation derivatives of a nucleic acid. Such derivatives can, for example, be modified in individual or several nucleotide positions by substitution, alteration, exchange, deletion or insertion. The modification or derivatization can, for example, be carried out by means of site directed mutagenesis. Such modifications can easily be carried out by a person skilled in the art (see e.g. Sambrook, J., et al., Molecular Cloning: A laboratory manual (1999) Cold Spring Harbor Laboratory Press, New York, USA; Hames, B. D., and Higgins, S. G., Nucleic acid hybridization—a practical approach (1985) IRL Press, Oxford, England).

Useful methods and techniques for carrying out the current invention are described in e.g. Ausubel, F. M. (ed.), Current Protocols in Molecular Biology, Volumes I to III (1997); Glover, N. D., and Hames, B. D., ed., DNA Cloning: A Practical Approach, Volumes I and II (1985), Oxford University Press; Freshney, R. I. (ed.), Animal Cell Culture—a practical approach, IRL Press Limited (1986); Watson, J. D., et al., Recombinant DNA, Second Edition, CHSL Press (1992); Winnacker, E. L., From Genes to Clones; N.Y., VCH Publishers (1987); Celis, J., ed., Cell Biology, Second Edition, Academic Press (1998); Freshney, R. I., Culture of Animal Cells: A Manual of Basic Technique, second edition, Alan R. Liss, Inc., N.Y. (1987).

The term “about” denotes a range of +/−20% of the thereafter following numerical value. In one embodiment the term about denotes a range of +/−10% of the thereafter following numerical value. In one embodiment the term about denotes a range of +/−5% of the thereafter following numerical value.

The term “antibody” is used herein in the broadest sense and encompasses various antibody structures, including but not limited to monoclonal antibodies, polyclonal antibodies, and multispecific antibodies (e.g., bispecific antibodies) so long as they exhibit the desired antigen-binding activity. For example, naturally occurring IgG antibodies are heterotetrameric glycoproteins of about 150,000 daltons, composed of two identical light chains and two identical heavy chains that are disulfide-bonded. From N- to C-terminus, each heavy chain has a variable domain (VH), also called a variable heavy domain or a heavy chain variable region, followed by three constant heavy domains (CH1, CH2, and CH3). Similarly, from N- to C-terminus, each light chain has a variable domain (VL), also called a variable light domain or a light chain variable region, followed by a constant light (CL) domain.

In certain embodiments, at least one domain of the fusion polypeptide is an antibody fragment. The term “antibody fragment” refers to a molecule other than an intact antibody that comprises a portion of an intact antibody that retains the ability to specifically bind to an antigen. Antibody fragments include, but are not limited to Fab, Fab′, Fab′-SH, F(ab′)2, Fv, single-chain Fab (scFab), single-chain variable fragments (scFv) and single domain antibodies (dAbs). For a review of certain antibody fragments, see Holliger and Hudson, Nature Biotechnology 23 (2005) 1126-1136.

In one embodiment, the antibody fragment is a Fab, Fab′, Fab′-SH, or F(ab′)2 fragment, in particular a Fab fragment. Papain digestion of intact antibodies produces two identical antigen-binding fragments, called “Fab” fragments containing each the heavy- and light-chain variable domains (VH and VL, respectively) and also the constant domain of the light chain (CL) and the first constant domain of the heavy chain (CH1). The term “Fab fragment” thus refers to an antibody fragment comprising a light chain comprising a VL domain and a CL domain, and a heavy chain fragment comprising a VH domain and a CH1 domain. Fab′ fragments differ from Fab fragments by the addition of residues at the carboxy terminus of the CH1 domain including one or more cysteines from the antibody hinge region. Fab′-SH are Fab′ fragments in which the cysteine residue(s) of the constant domains bear a free thiol group. Pepsin treatment yields an F(ab′)2 fragment that has two antigen-binding sites (two Fab fragments) and a part of the Fc region. For discussion of Fab and F(ab′)2 fragments comprising salvage receptor binding epitope residues and having increased in vivo half-life, see U.S. Pat. No. 5,869,046.

In another embodiment, the antibody fragment is a diabody, a triabody or a tetrabody. Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

In a further embodiment, the antibody fragment is a single chain Fab fragment. A “single chain Fab fragment” or “scFab” is a polypeptide consisting of an antibody heavy chain variable domain (VH), an antibody heavy chain constant domain 1 (CH1), an antibody light chain variable domain (VL), an antibody light chain constant domain (CL) and a peptidic linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL. In particular, said peptidic linker is a polypeptide of at least 30 amino acids, preferably between 32 and 50 amino acids. Said single chain Fab fragments are stabilized via the natural disulfide bond between the CL domain and the CH1 domain. In addition, these single chain Fab fragments might be further stabilized by generation of interchain disulfide bonds via insertion of cysteine residues (e.g., position 44 in the variable heavy chain and position 100 in the variable light chain according to Kabat numbering).

In one embodiment, the antibody fragment is a Fab fragment with a domain crossover. The term “domain crossover” as used herein denotes that in a pair of an antibody heavy chain VH-CH1 fragment and its corresponding cognate antibody light chain, i.e. in an antibody binding arm (i.e. in the Fab fragment), the domain sequence deviates from the natural sequence in that at least one heavy chain domain is substituted by its corresponding light chain domain and vice versa. There are three general types of domain crossovers, (i) the crossover of the CH1 and the CL domains, which leads to domain crossover light chain with a VL-CH1 domain sequence and a domain crossover heavy chain fragment with a VH-CL domain sequence (or a full length antibody heavy chain with a VH-CL-hinge-CH2-CH3 domain sequence), (ii) the domain crossover of the VH and the VL domains, which leads to domain crossover light chain with a VH-CL domain sequence and a domain crossover heavy chain fragment with a VL-CH1 domain sequence, and (iii) the domain crossover of the complete light chain (VL-CL) and the complete VH-CH1 heavy chain fragment (“Fab crossover”), which leads to a domain crossover light chain with a VH-CH1 domain sequence and a domain crossover heavy chain fragment with a VL-CL domain sequence (all aforementioned domain sequences are indicated in N-terminal to C-terminal direction).

As used herein the term “replaced by each other” with respect to corresponding heavy and light chain domains refers to the aforementioned domain crossovers. As such, when CH1 and CL domains are “replaced by each other” it is referred to the domain crossover mentioned under item (i) and the resulting heavy and light chain domain sequence. Accordingly, when VH and VL are “replaced by each other” it is referred to the domain crossover mentioned under item (ii); and when the CH1 and CL domains are “replaced by each other” and the VH1 and VL domains are “replaced by each other” it is referred to the domain crossover mentioned under item (iii). Bispecific antibodies including domain crossovers are reported, e.g. in WO 2009/080251, WO 2009/080252, WO 2009/080253, WO 2009/080254 and Schaefer, W., et al, Proc. Natl. Acad. Sci USA 108 (2011) 11187-11192.

Fusion polypeptides according to the current invention can also comprises Fab fragments including a domain crossover of the CH1 and the CL domains as mentioned under item (i) above, or a domain crossover of the VH and the VL domains as mentioned under item (ii) above. The Fab fragments specifically binding to the same antigen(s) are constructed to be of the same domain sequence. Hence, in case more than one Fab fragment with a domain crossover is contained in the multispecific antibody, said Fab fragment(s) specifically bind to the same antigen.

An “isolated” fusion polypeptide is one, which has been separated from a component of its natural environment. In some embodiments, a fusion polypeptide is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman, S. et al., J. Chromatogr. B 848 (2007) 79-87.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

The term “N-linked oligosaccharide” denotes oligosaccharides that are linked to the peptide backbone at an asparagine amino acid residue, by way of an asparagine-N-acetyl glucosamine linkage. N-linked oligosaccharides are also called “N-glycans.”

All N-linked oligo saccharides have a common pentasaccharide core of Man3GlcNAc2. They differ in the presence of, and in the number of branches (also called antennae) of peripheral sugars such as N-acetyl glucosamine, galactose, N-acetyl galactosamine, fucose and sialic acid. Optionally, this structure may also contain a core fucose molecule and/or a xylose molecule. N-linked oligosaccharides are attached to a nitrogen of asparagine or arginine side-chains. N-glycosylation motifs, i.e. N-glycosylation sites, comprise an Asn-X-Ser/Thr consensus sequence, where X is any amino acid except proline. Thus, an amino acid residue in an N-glycosylation site can be any amino acid residue in the Asn-X-Ser/Thr consensus sequence, where X is any amino acid except proline. In one embodiment is the amino acid residue in an N-glycosylation site Asn, Ser or Thr.

The term “O-linked oligosaccharide” denotes oligosaccharides that are linked to the peptide backbone at a threonine or serine amino acid residue. In one embodiment is the amino acid residue in an O-glycosylation site Ser or Thr.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt, T. J. et al. Kuby Immunology, 6th ed., W.H. Freeman and Co., N.Y. (2007), page 91) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano, S. et al., J. Immunol. 150 (1993) 880-887; Clackson, T. et al., Nature 352 (1991) 624-628).

As used herein, the term “post-translational modification” generally denotes modifications made to polypeptides after translation. Non-limiting examples of post-translational modification are the attachment of functional groups such as acetate, phosphate, lipids, or carbohydrates to the polypeptide in a cell (i.e. in vivo).

In more detail, the term “post-translational modification” denotes a covalent modification of amino acid residues within a polypeptide following biosynthesis. Post-translational modifications can occur on the amino acid side chains by modifying an existing functional group or by introducing a new one. Known post-translational modifications of the different proteinogenic amino acids are e.g.
Ala: N-acetylation
Arg: deimination, methylation
Asn: deamidation, N-linked glycosylation
Asp: isomerization
Cys: disulfide-bond formation, oxidation, N-acetylation
Gln: cyclization
Glu: cyclization, gamma-carboxylation
Gly: N-myristoylation, N-acetylation
His: phosphorylation
Lys: acetylation, ubiquitination, methylation, hydroxylation
Met: N-acetylation, oxidation
Pro: hydroxylation and subsequent further modification
Ser: phosphorylation, O-linked glycosylation
Thr: phosphorylation, O-linked glycosylation, N-acetylation
Trp: oxidation
Tyr: phosphorylation
Val: N-acetylation.

The term “glycosylation” as used herein denotes the covalent linking of one or more carbohydrate moiety(ies) to an amino acid residue within a polypeptide. Typically, glycosylation is a post-translational event which can occur within the intracellular milieu of a cell or cellular extract. The term glycosylation includes, for example, addition of one or more carbohydrate moieties at a consensus site for glycosylation. One example of glycosylation involves the addition of one or more xylose residues to a polypeptide. For example, the consensus sequence for xylose addition comprises the sequence [D/E GSG D/E]. The term “O-glycosylation” denotes the covalent linkage of one or more carbohydrate moiety(ies) to an oxygen atom in the side chain of an amino acid residue in a polypeptide, such as to the oxygen of serine or threonine.

The term “phosphorylation” denotes the covalent linking of a phosphate group (PO₄) to an amino acid residue within a polypeptide. Typically, phosphorylation is a post-translational event which can occur within the intracellular milieu of a cell or cellular extract. The term phosphorylation includes, for example, addition of a phosphate group to the free hydroxyl group of serine.

Homogeneity of polypeptides is important if those polypeptides are to be used therapeutically. Surprisingly, as demonstrated herein, alteration of the amino acid sequence of peptidic linkers in a fusion polypeptide has been found to reduce or eliminate the post-translational modification at the C-terminus of said peptidic linker. The reduction or even elimination of post-translationally added modifications improves the homogeneity of the polypeptide.

As used herein, the term “polypeptide” denotes a polymer comprising ten or more (up to 650) naturally occurring amino acid residues conjugated to each other by peptide bonds.

The term “amino acid” as used herein includes alanine (Ala (three letter code) or A (one letter code)), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I): leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V).

As used herein the term “peptidic linker” denotes a synthetic amino acid sequence that connects or links two polypeptide sequences, e.g., that fuses two polypeptide domains together. The peptidic linker by itself then represents a third domain. Thus, a fusion polypeptide comprising a peptidic linker according to the invention comprises at least three domains: the first (first polypeptide) domain, the second (peptidic linker) domain and the third (second polypeptide) domain. As used herein the term “synthetic” denotes amino acid sequences that are not naturally occurring. Thus, in one embodiment the peptidic linker according to the current invention is a synthetic peptidic linker.

Peptidic linkers of the invention connect two amino acid sequences via peptide bonds. In one embodiment of the aspects of the invention, the peptidic linker of the invention connects a first biologically active polypeptide (first domain) to a second polypeptide (third domain) in a linear sequence. In another embodiment of the aspects of the invention, the peptidic linker connects two biologically active polypeptides.

In the context of fusion polypeptides, a “linear sequence” or a “sequence” denotes the order of amino acids in a fusion polypeptide in an amino to carboxyl terminal direction (N- to C-terminal direction) in which residues that neighbor each other in the sequence are contiguous in the primary structure of the polypeptide.

As used herein, the terms “conjugated”, “linked”, “fused”, or “fusion” can be used interchangeably. These terms refer to the joining together of two or more polypeptides or domains by recombinant means. As used herein, the term “genetically fused,” “genetically linked” or “genetic fusion” denotes the co-linear, covalent linkage or attachment of two or more polypeptides via their individual peptide backbones, through recombinant expression of a single nucleic acid molecule encoding the fusion polypeptide in a eukaryotic cell. Such genetic fusion results in the expression of a single contiguous genetic sequence. Preferred genetic fusions are in frame, i.e., two or more open reading frames (ORFs) are fused to form a continuous longer ORF, in a manner that maintains the correct reading frame of the original ORFs. Thus, the resulting recombinant fusion polypeptide is a single polypeptide containing two or more polypeptide domains that correspond to the polypeptides encoded by the original ORFs (which segments are not naturally conjugated to each other).

The peptidic linkers of the current invention differ from the traditional Gly/Ser (GS)-peptidic linkers of the art in that the presently claimed peptidic linkers lack at least the C-terminal serine amino acid residue and in that the next thereafter following amino acid residue shall be no serine, threonine or proline amino acid residue.

As used herein, the term “gly-ser linker” or “GS-peptidic linker” denotes a polypeptide that consists of glycine and serine residues. An exemplary gly/ser-peptidic linker comprises the amino acid sequence (Gly₄Ser)ₙ. In one embodiment of all aspects of the invention, the peptidic linker comprises or consists of a GS-peptidic linker with one or more amino acid substitutions, deletions, and/or additions and which lacks a C-terminal serine residue.

In one embodiment of the aspects of the invention, the fusion polypeptide of the invention is a “chimeric” polypeptide. Such chimeric fusion polypeptides comprise a first amino acid sequence (first domain) linked to a second amino acid sequence (third domain) to which it is not naturally linked in nature by means of a peptidic linker according to the current invention (second domain). The amino acid sequences of the first and the third domain polypeptides, which may exist in separate proteins or they may exist in the same protein but apart from each other, are brought together in the fusion polypeptide in a new arrangement. A chimeric fusion polypeptide may be created, for example, by creating and translating a polynucleotide in which the domains are encoded in the desired relationship. Exemplary chimeric fusion polypeptides include fusion polypeptides comprising the peptidic linkers of the invention.

Fusion polypeptides which comprise a peptidic linker of the invention may be either monomeric or multimeric. For example, in one embodiment, a fusion polypeptide of the invention is a dimer or a tetramer.

In one embodiment of the aspects of the invention, the dimer of the fusion polypeptide of the invention is a homodimer, comprising two identical monomeric fusion polypeptides of the invention.

In another embodiment of the aspects of the invention, the dimer of the fusion polypeptide of the invention is a heterodimer, comprising two non-identical monomeric subunits whereof at least one is a fusion polypeptide according to the invention.

In another embodiment of the aspects of the invention, the tetramer of the fusion polypeptide of the invention is a heterotetramer, comprising at least three non-identical monomeric subunits whereof at least one is a fusion polypeptide according to the invention.

In addition to comprising one or more peptidic linkers according to the current invention, in certain embodiments, a fusion polypeptide may comprise one or more traditional GS-peptidic linkers at other locations within the fusion polypeptide.

The fusion polypeptide according to the aspects of the current invention comprises at least one biologically active moiety. A biologically active moiety refers to a moiety capable of one or more of: localizing or targeting a molecule to a desired site or cell, performing a function, performing an action or a reaction in a biological context. For example, the term “biologically active moiety” refers to biologically active molecules or portions thereof which bind to components of a biological system (e.g., proteins in sera or on the surface of cells or in cellular matrix) and which binding results in a biological effect (e.g., as measured by a change in the active moiety and/or the component to which it binds (e.g., a cleavage of the active moiety and/or the component to which it binds, the transmission of a signal, or the augmentation or inhibition of a biological response in a cell or in a subject)).

Exemplary biologically active moieties comprise, e.g., an antigen binding fragment of an antibody molecule or portion thereof (e.g., F(ab), scFv, a VH domain, or a VL domain) (e.g., to act as a targeting moiety or to impart, induce or block a biological response), a ligand binding portion of a receptor or a receptor binding portion of a ligand, and Fc-region polypeptide, a complete Fc-region, an scFc domain, an enzyme, etc. In addition, as used herein, the term “biologically active moiety” includes, for example, moieties which may not have activity when present alone in monomeric form, but which have a biological activity when paired with a second moiety in the context of a dimeric molecule.

In one embodiment of the aspects of the invention, the fusion polypeptide of the invention comprises in at least one of its fused domains a binding site or the binding site is obtained by the fusion of the domains in the fusion polypeptide. The terms “binding domain” or “binding site”, as used herein, denote the portion, region, or site of polypeptide that mediates specific interaction with a target molecule (e.g. an antigen, ligand, receptor, substrate or inhibitor). Exemplary binding domains include an antigen binding site (e.g. a VH or VL domain or a pair thereof) or molecules comprising such a binding site (e.g. an antibody), a receptor binding domain of a ligand, a ligand binding domain of a receptor or a catalytic domain.

In one embodiment of the aspects of the invention, the fusion polypeptide comprises (has) at least one binding domain specifically binding to a target. In one embodiment of the aspects of the invention, the binding domain comprises or consists of an antigen binding site (e.g. comprising a variable heavy chain domain and variable light chain domain or at least six CDRs from an antibody or at least a functional part thereof comprising only an isolated VH or VL or three CDRs. In one embodiment of the aspects of the invention, the binding domain serves as a targeting moiety.

In one embodiment of the aspects of the invention, the fusion polypeptides are modified antibody chains or modified antibodies. As used herein, the term “modified antibody chain” and “modified antibody” includes synthetic forms of antibody chains or antibodies which are altered such that they are not naturally occurring, e.g., by changing the natural domain structure, sequence or number. For example, comprising heavy chain molecules joined to scFv molecules and the like. In addition, the term “modified antibody” includes multivalent forms of antibodies (e.g., trivalent, tetravalent, etc., antibodies that bind to three or more copies of the same antigen).

In one embodiment of all aspects of the invention, the fusion polypeptide comprises an Fc-region, or domain thereof or an Fc-region polypeptide.

The term “antibody-dependent cellular cytotoxicity (ADCC)” is a function mediated by Fc-receptor binding and refers to lysis of target cells by an antibody as reported herein in the presence of effector cells. ADCC is measured in one embodiment by the treatment of a preparation of CD19 expressing erythroid cells (e.g. K562 cells expressing recombinant human CD19) with an antibody comprising the fusion polypeptide as reported herein in the presence of effector cells such as freshly isolated PBMC (peripheral blood mononuclear cells) or purified effector cells from buffy coats, like monocytes or NK (natural killer) cells. Target cells are labeled with 51Cr and subsequently incubated with the antibody. The labeled cells are incubated with effector cells and the supernatant is analyzed for released 51Cr. Controls include the incubation of the target endothelial cells with effector cells but without the antibody comprising the fusion polypeptide. The capacity of the antibody to induce the initial steps mediating ADCC is investigated by measuring their binding to Fcγ receptors expressing cells, such as cells, recombinantly expressing FcγRT and/or FcγRIIA or NK cells (expressing essentially FcγRIIIA). In one embodiment binding to FcγR on NK cells is measured.

“Effector functions” refer to those biological activities attributable to the Fc-region of an antibody, which vary with the antibody class. Examples of antibody effector functions include: C1q binding and complement dependent cytotoxicity (CDC); Fc-receptor binding; antibody-dependent cell-mediated cytotoxicity (ADCC); phagocytosis; down regulation of cell surface receptors (e.g. B-cell receptor); and B-cell activation.

Fc-receptor binding dependent effector functions can be mediated by the interaction of the Fc-region of an antibody with Fc-receptors (FcRs), which are specialized cell surface receptors on hematopoietic cells. Fc-receptors belong to the immunoglobulin superfamily, and have been shown to mediate both the removal of antibody-coated pathogens by phagocytosis of immune complexes, and the lysis of erythrocytes and various other cellular targets (e.g. tumor cells) coated with the corresponding antibody, via antibody dependent cell mediated cytotoxicity (ADCC) (see e.g. Van de Winkel, J. G. and Anderson, C. L., J. Leukoc. Biol. 49 (1991) 511-524). FcRs are defined by their specificity for immunoglobulin isotypes: Fc-receptors for IgG antibodies are referred to as FcγR. Fc-receptor binding is described e.g. in Ravetch, J. V. and Kinet, J. P., Annu. Rev. Immunol. 9 (1991) 457-492; Capel, P. J., et al., Immunomethods 4 (1994) 25-34; de Haas, M., et al., J. Lab. Clin. Med. 126 (1995) 330-341; and Gessner, J. E., et al., Ann. Hematol. 76 (1998) 231-248.

Cross-linking of receptors for the Fc-region of IgG antibodies (FcγR) triggers a wide variety of effector functions including phagocytosis, antibody-dependent cellular cytotoxicity, and release of inflammatory mediators, as well as immune complex clearance and regulation of antibody production. In humans, three classes of FcγR have been characterized, which are:
FcγRI (CD64) binds monomeric IgG with high affinity and is expressed on macrophages, monocytes, neutrophils and eosinophils. Modification in the Fc-region IgG at least at one of the amino acid residues E233-G236, P238, D265, N297, A327 and P329 (numbering according to EU index of Kabat) reduce binding to FcγRI. IgG2 residues at positions 233-236, substituted into IgG1 and IgG4, reduced binding to FcγRI by 10³-fold and eliminated the human monocyte response to antibody-sensitized red blood cells (Armour, K. L., et al., Eur. J. Immunol. 29 (1999) 2613-2624),
FcγRII (CD32) binds complexed IgG with medium to low affinity and is widely expressed. This receptor can be divided into two sub-types, FcγRIIA and FcγRIIB. FcγRIIA is found on many cells involved in killing (e.g. macrophages, monocytes, neutrophils) and seems able to activate the killing process. FcγRIIB seems to play a role in inhibitory processes and is found on B-cells, macrophages and on mast cells and eosinophils. On B-cells it seems to function to suppress further immunoglobulin production and isotype switching to, for example, the IgE class. On macrophages, FcγRIIB acts to inhibit phagocytosis as mediated through FcγRIIA. On eosinophils and mast cells the B-form may help to suppress activation of these cells through IgE binding to its separate receptor. Reduced binding for FcγRIIA is found e.g. for antibodies comprising an IgG Fc-region with mutations at least at one of the amino acid residues E233-G236, P238, D265, N297, A327, P329, D270, Q295, A327, R292, and K414 (numbering according to EU index of Kabat),
FcγRIII (CD16) binds IgG with medium to low affinity and exists as two types. FcγRIIIA is found on NK cells, macrophages, eosinophils and some monocytes and T cells and mediates ADCC. FcγRIIIB is highly expressed on neutrophils. Reduced binding to FcγRIIIA is found e.g. for antibodies comprising an IgG Fc-region with mutation at least at one of the amino acid residues E233-G236, P238, D265, N297, A327, P329, D270, Q295, A327, 5239, E269, E293, Y296, V303, A327, K338 and D376 (numbering according to EU index of Kabat).

Mapping of the binding sites on human IgG1 for Fc-receptors, the above mentioned mutation sites and methods for measuring binding to FcγRI and FcγRIIA are described in Shields, R. L., et al., J. Biol. Chem. 276 (2001) 6591-6604.

An “effective amount” of an agent, e.g., a pharmaceutical formulation, refers to an amount effective, at dosages and for periods of time necessary, to achieve the desired therapeutic or prophylactic result.

The term “Fc-receptor” as used herein refers to activation receptors characterized by the presence of a cytoplasmatic ITAM sequence associated with the receptor (see e.g. Ravetch, J. V. and Bolland, S., Annu. Rev. Immunol. 19 (2001) 275-290). Such receptors are FcγRI, FcγRIIA and FcγRIIIA. The term “no binding of FcγR” denotes that at an antibody concentration of 10 μg/ml the binding of an antibody as reported herein to NK cells is 10% or less of the binding found for anti-OX40L antibody LC.001 as reported in WO 2006/029879.

The term “Fc-region” herein is used to define a C-terminal region of an immunoglobulin heavy chain that contains at least a portion of the constant region. The term includes native sequence Fc-regions and variant Fc-regions. In one embodiment, a human IgG heavy chain Fc-region extends from Cys226, or from Pro230, to the carboxyl-terminus of the heavy chain. However, the C-terminal lysine (Lys447) of the Fc-region may or may not be present. An Fc-region is a dimer of two Fc-region polypeptides.

The Fc-region of an antibody is directly involved in complement activation, C1q binding, C3 activation and Fc-receptor binding. While the influence of an antibody on the complement system is dependent on certain conditions, binding to C1q is caused by defined binding sites in the Fc-region. Such binding sites are known in the state of the art and described e.g. by Lukas, T. J., et al., J. Immunol. 127 (1981) 2555-2560; Brunhouse, R. and Cebra, J. J., Mol. Immunol. 16 (1979) 907-917; Burton, D. R., et al., Nature 288 (1980) 338-344; Thommesen, J. E., et al., Mol. Immunol. 37 (2000) 995-1004; Idusogie, E. E., et al., J. Immunol. 164 (2000) 4178-4184; Hezareh, M., et al., J. Virol. 75 (2001) 12161-12168; Morgan, A., et al., Immunology 86 (1995) 319-324; and EP 0 307 434. Such binding sites are e.g. L234, L235, D270, N297, E318, K320, K322, P331 and P329 (numbering according to EU index of Kabat). Antibodies of subclass IgG1, IgG2 and IgG3 usually show complement activation, C1q binding and C3 activation, whereas IgG4 do not activate the complement system, do not bind C1q and do not activate C3.

An “Fc-region of an antibody” is a term well known to the skilled artisan and defined on the basis of papain cleavage of antibodies. In one embodiment the Fc-region is a human Fc-region. In one embodiment the Fc-region is of the human IgG4 subclass comprising the mutations S228P and/or L235E (numbering according to EU index of Kabat). In one embodiment the Fc-region is of the human IgG1 subclass comprising the mutations L234A, L235A and optionally P329G (numbering according to EU index of Kabat).

As used herein, the term “Fc-region polypeptide” denotes the portion of a single immunoglobulin heavy chain beginning in the hinge region just upstream of the papain cleavage site (i.e. residue 216 in IgG, taking the first residue of heavy chain constant region to be 114) and ending at the C-terminus of the antibody. Accordingly, a complete Fc-region polypeptide comprises at least a hinge domain, a CH2 domain, and a CH3 domain. As used herein, the term “Fc-region” denotes the dimerized Fc-region polypeptide which resemble the Fc-region of native antibodies (e.g., whether made in the traditional two polypeptide chain format or as a single chain Fc region).

As used herein, the term “Fc-region polypeptide portion” includes an amino acid sequence of an Fc-region polypeptide or derived from an Fc-region polypeptide. In certain embodiments, an Fc-region polypeptide portion comprises at least one of: a hinge (e.g., upper, middle, and/or lower hinge region) domain, a CH2 domain, a CH3 domain, a CH4 domain, or a variant, portion, or fragment thereof. In other embodiments, an Fc-region polypeptide portion comprises a complete Fc-region polypeptide (i.e., a hinge domain, a CH2 domain, and a CH3 domain). In one embodiment, a Fc-region polypeptide portion comprises a hinge domain (or portion thereof) fused to a CH3 domain (or portion thereof). In another embodiment, an Fc-region polypeptide portion comprises a CH2 domain (or portion thereof) fused to a CH3 domain (or portion thereof). In another embodiment, an Fc-region polypeptide portion consists of a CH3 domain or portion thereof. In another embodiment, an Fc-region polypeptide portion consists of a hinge domain (or portion thereof) and a CH3 domain (or portion thereof). In another embodiment, a Fc-region polypeptide portion consists of a CH2 domain (or portion thereof) and a CH3 domain. In another embodiment, a Fc-region polypeptide portion consists of a hinge domain (or portion thereof) and a CH2 domain (or portion thereof). In one embodiment, an Fc-region polypeptide portion lacks at least a portion of a CH2 domain (e.g., all or part of a CH2 domain).

In one embodiment, an Fc-region polypeptide comprises at least the portion of an Fc-region known in the art to be required for FcRn binding, referred to herein as a neonatal receptor (FcRn) binding partner. An FcRn binding partner is a molecule or portion thereof that can be specifically bound by the FcRn receptor with consequent active transport by the FcRn receptor of the FcRn binding partner. Specifically bound refers to two molecules forming a complex that is relatively stable under physiologic conditions. Specific binding is characterized by a high affinity and a low to moderate capacity as distinguished from nonspecific binding which usually has a low affinity with a moderate to high capacity.

Typically, binding is considered specific when the affinity constant KA is higher than 10⁶ M⁻¹, or more preferably higher than 10⁸ M⁻¹.

The FcRn receptor has been isolated from several mammalian species including humans. The sequences of the human FcRn, monkey FcRn, rat FcRn, and mouse FcRn are known (Story, et al., J. Exp. Med. 180 (1994) 2377). The FcRn receptor binds IgG (but not other immunoglobulin classes such as IgA, IgM, IgD, and IgE) at relatively low pH, actively transports the IgG transcellularly in a luminal to serosal direction, and then releases the IgG at relatively higher pH found in the interstitial fluids.

FcRn binding partners encompass molecules that can be specifically bound by the FcRn receptor including whole IgG, the Fc-region of IgG, and other fragments that include the complete binding region of the FcRn receptor. The part of the Fc-region of IgG that binds to the FcRn receptor has been described based on X-ray crystallography (Burmeister et al. Nature 372 (1994) 379). The major contact area of the Fc with the FcRn is near the junction of the CH2 and CH3 domains. Fc-FcRn contacts are all within a single Ig heavy chain polypeptide. The FcRn binding partners include whole IgG, the Fc-region of IgG, the single Fc-region polypeptide and other fragments of IgG that include the complete binding region of FcRn. The major contact sites include amino acid residues 248, 250-257, 272, 285, 288, 290-291, 308-311, and 314 of the CH2 domain and amino acid residues 385-387, 428, and 433-436 of the CH3 domain.

The fusion polypeptides of the invention comprise at least one peptidic linker of the invention. In one embodiment, a fusion polypeptide comprises between 1 and 10 peptidic linkers, inclusive. In one embodiment, two or more peptidic linkers are present in a fusion polypeptide of the invention. In another embodiment, a fusion polypeptide of the invention comprises 1, 2, 3, or 4 peptidic linkers of the invention.

Peptidic linkers of the invention may occur one time at a given position, or may occur multiple times at different positions within the same fusion polypeptide.

The peptidic linkers of the invention are modified from those in the art such that the C-terminal serine amino acid is removed or replaced by a glycine residue with the proviso that the next following amino acid residue from the fused polypeptide is not a serine, threonine or proline amino acid residue.

Peptidic linkers of the invention can be of varying lengths. In one embodiment, a peptidic linker of the invention is from about 6 to about 75 amino acids in length. In another embodiment, a peptidic linker of the invention is from about 6 to about 50 amino acids in length. In another embodiment, a peptidic linker of the invention is from about 10 to about 40 amino acids in length. In another embodiment, a peptidic linker of the invention is from about 15 to about 35 amino acids in length. In another embodiment, a peptidic linker of the invention is from about 15 to about 20 amino acids in length. In another embodiment, a peptidic linker of the invention is about 15 amino acids in length.

The position(s) of a peptidic linker of the invention may vary depending on the domains of the fusion polypeptide that it connects. Although different specific examples of fusion polypeptides comprising peptidic linkers are disclosed herein, it will be understood that peptidic linkers may be positioned at least wherever peptidic linkers are presently positioned in recombinant fusion polypeptides. Peptidic linkers are so frequently used in protein engineering that they have become standard assembly parts in synthetic biology.

Some examples of current, art recognized uses for peptidic linkers include uses in: scFv molecules (Freund et al., FEBS 320 (1993) 97), single chain immunoglobulin molecules (Shun et al., Proc. Natl. Acad. Sci. USA 90 (1993) 7995), minibodies (Hun et al., Cancer Res. 56 (1996) 3055), CH2 domain deleted antibodies (Mueller et al., Proc. Natl. Acad. Sci. USA. 87 (1990) 5702), single chain bispecific antibodies (Schertz et al., Cancer Res. 65 (2005) 2882), full-length IgG-like bispecific antibodies (Marvin et al., Acta Pharm. Sin. 26 (2005) 649, Michelson et al., MAbs 1 (2009) 128, Routt et al., Prot. Eng. Des. Sell. 23 (2006) 221), scFv fusion proteins (Degree et al., Brit. J. Canc. 86 (2002) 811), developing protein-fragment complementation assays (Remy et al., BioTechniques 42 (2008) 137), and in scFc molecules (WO 2008/143954).

Peptidic linkers may be attached to the N- or to the C-terminus (or both) of polypeptides which they are used to fuse with other polypeptides.

In another embodiment, a peptidic linker of the invention can be used to genetically fuse two biologically active polypeptides (each is a domain), wherein each polypeptide has biological activity alone.

In another embodiment, a peptidic linker of the invention is used to fuse two polypeptides to each other, wherein neither polypeptide has biological activity alone, but when genetically fused, is biologically active.

For example, in one embodiment, a peptidic linker of the invention can be used to genetically fuse the VH and VL in an scFv molecule: A-L-B, wherein A is VH or VL, B is VH or VL, and L is a peptidic linker according to the invention or A-L-B-L, wherein A is VH or VL, B is VH or VL, and L is a peptidic linker according to the invention.

In another embodiment, a peptidic linker can be used to genetically fuse a biologically active polypeptide to a complete Fc-region, an Fc-region polypeptide, an Fc-region polypeptide portion, or an scFc region: C-L-Fc, wherein C is a biologically active polypeptide, L is a peptidic linker according to the invention, and

Fc is an Fc-region (e.g., single chain or traditional two polypeptide chain), Fc-region polypeptide, an Fc-region polypeptide portion, or an scFc region. For example, in one embodiment, C comprises a scFv molecule (e.g., comprising VH-L-VL or VL-L-VH, where L is a peptidic linker) and Fc consists of a Fc-region polypeptide (hinge-CH2-CH3 domain) or an scFc region, thus forming a scFv-Fc fusion protein or a scFv-scFc fusion protein. In another embodiment, C comprises an scFv molecule (e.g., comprising VH-L-VL or VL-L-VH, where L is a peptidic linker and Fc is a CH3 domain, thus forming a minibody. In another embodiment, C comprises two tandem scFv molecules and an Fc-region polypeptide portion which is a CH3 domain, thereby forming a tetravalent minibody.

A tetravalent minibody may also be formed using the format: A-L-B-L-Fc-L-A-L-B, where A and B are each one of a VH or VL domain, L is a peptidic linker according to the invention and Fc is a CH3 domain or an scFc region.

In another embodiment, a fusion polypeptide of the invention may have the format: D-L-A-L-B, where D is a complete antibody molecule, L is a peptidic linker according to the invention, and A and B are each a VH or VL domain. Such a construct yields a C-terminal tetravalent antibody molecule.

In another embodiment, a fusion polypeptide of the invention may have the format: A-L-B-L-D, where D is a complete antibody molecule, L is a peptidic linker according to the invention, and A and B are each a VH or VL domain. Such a construct yields an N-terminal tetravalent antibody molecule. In such a construct, the A-L-B (scFv) portion of the molecule may be genetically fused to either the light chain or the heavy chain variable region.

In another embodiment, a peptidic linker of the invention can be used to fuse a CH3 domain to a hinge region. In another embodiment, a peptidic linker of the invention can be used to fuse a CH3 domain to a CH1 domain. In still another embodiment, a peptidic linker according to the invention can act as a spacer between the hinge region and a CH2 domain. Preferred locations for peptidic linker according to the invention are between the Fc-region or Fc-region polypeptide and a scFv or Fab.

Where more than one binding site is included in a polypeptide, it will be understood that such molecules may be monospecific or multispecific, i.e., the binding sites may be the same or may be different.

Peptidic linkers can be introduced into polypeptide sequences using techniques known in the art. Modifications can be confirmed by DNA sequence analysis. Plasmid DNA can be used to transform host cells for stable production of the polypeptides produced.

The fusion polypeptides of the invention comprise at least one biologically active polypeptide (domain). Such a polypeptide can be biologically active as a single molecule or may require association with another polypeptide (e.g., when linked with a second polypeptide via a peptidic linker or when present in a polypeptide dimer).

In one embodiment, the fusion polypeptides of the invention comprise only one biologically active polypeptide (domain) (creating a molecule which is monomeric with regard to the biologically active polypeptide, but which may be monomeric or dimeric with regard to the number of polypeptide chains). In another embodiment, a fusion polypeptide of the invention comprises more than one biologically active polypeptide (domain), e.g., 2, 3, 4, 5, 6, 7, 8, 9, 10, or more biologically active polypeptides.

As used herein, the term “biologically active polypeptide” is not meant to include chemical effector moieties that may be added to a polypeptide (e.g., toxic moieties, detectable moieties and the like) by chemical means.

In one embodiment of the invention, a biologically active polypeptide is operably linked via a peptidic linker according to the invention to the N-terminus of an Fc-region polypeptide, or portion thereof. In another embodiment, the biologically active polypeptide is operably linked via a peptidic linker according to the invention to the C-terminus of an Fc-region polypeptide.

In other embodiments, two or more biologically active polypeptides are linked to each other (e.g. via a peptidic linker according to the invention) in series. In one embodiment, the tandem array of biologically active polypeptides is operably linked via a peptidic linker according to the invention to either the C-terminus or the N-terminus of an Fc-region polypeptide or portion thereof.

In one embodiment, a fusion polypeptide of the invention comprises at least one of an antigen binding site (e.g., an antigen binding site of an antibody, antibody variant, or antibody fragment), a receptor binding portion of ligand, or a ligand binding portion of a receptor.

In one embodiment, a biologically active polypeptide comprises an antigen binding site.

In certain embodiments, the fusion polypeptides of the invention have at least one binding site specific for a target molecule which mediates a biological effect. In one embodiment, the binding site modulates cellular activation or inhibition (e.g., by binding to a cell surface receptor and resulting in transmission of an activating or inhibitory signal). In one embodiment, the binding site is capable of initiating transduction of a signal which results in death of the cell (e.g., by a cell signal induced pathway, by complement fixation or exposure to a payload (e.g., a toxic payload) present on the binding molecule), or which modulates a disease or disorder in a subject (e.g., by mediating or promoting cell killing, by promoting lysis of a fibrin clot or promoting clot formation, or by modulating the amount of a substance which is bioavailable (e.g., by enhancing or reducing the amount of a ligand such as TNF in the subject)). In another embodiment, the fusion polypeptides of the invention have at least one binding site specific for an antigen targeted for reduction or elimination, e.g., a cell surface antigen or a soluble antigen.

In another embodiment, binding of the fusion polypeptides of the invention to a target molecule (e.g. antigen) results in the reduction or elimination of the target molecule, e.g., from a tissue or from circulation.

In another embodiment, a fusion polypeptide has at least one binding site specific for a target molecule and can be used to detect the presence of the target molecule (e.g., to detect a contaminant or diagnose a condition or disorder).

In yet another embodiment, a fusion polypeptide of the invention comprises at least one binding site that targets the molecule to a specific site in a subject (e.g., to a tumor cell, an immune cell, or blood clot).

In certain embodiments, the fusion polypeptides of the invention may comprise two or more biologically active polypeptides. In one embodiment, the biologically active polypeptides are identical. In another embodiment, the biologically active polypeptides are different.

In certain particular embodiments, the fusion polypeptide of the invention is multispecific, e.g., has at least one binding site that binds to a first molecule or epitope of a molecule and at least one second binding site that binds to a second molecule or to a second epitope of the first molecule. Multispecific binding molecules of the invention may comprise at least two binding sites. In certain embodiments, at least one binding site of a multispecific binding molecule of the invention is an antigen binding region of an antibody or an antigen binding fragment thereof (e.g. an antibody or antigen binding fragment)

The following examples and figures are provided to aid the understanding of the present invention, the true scope of which is set forth in the appended claims. It is understood that modifications can be made in the procedures set forth without departing from the spirit of the invention.

### EXAMPLES OVERVIEW

Fusion protein 1—HC1-F:

Fusion protein 1 comprises two peptidic linkers connecting in N- to C-terminal direction a first non-IgG protein with a second non-IgG protein and an IgG heavy chain domain. It has been found that fusion protein 1 is with phosphorylation of the C-terminal terminal serine residue in the linker GGGGSGGGGSRE SEQ ID NO: 09 (cf FIGS. 1-8). Beside phosphorylation also xylosylation is present. Said phosphorylation results in a mass shift of +79 Da. Said xylosylation results in a mass shift of +132 Da. FIG. 1 shows the deconvoluted total mass spectrum of the deglycosylated and reduced HC1-F. In addition to the expected HC1-F, another peak evidencing the presence of an intense +79 Da product variant of HC1-F can be seen. That the modification is present in the linker of SEQ ID NO: 09 was verified by subjecting a tryptic digest of HC1-F to LC-MS analysis. FIG. 3 shows the extracted ion current (EIC) chromatograms (z=2 and 3) of (A) the unmodified (elution time 36.5 min) and (B) the +79.97 Da modified (elution time 37.6 min) tryptic glycine-serine linker peptides (X9GGGGSGGGGSR; SEQ ID NO: 07) of HC1-F. By using ion trap MS/MS analysis with collision induced dissociation of the triple protonated unmodified and the +79.97 Da modified tryptic digested glycine-serine linker peptide of HC1-F, as well as MS/MS analysis with electron-transfer/higher-energy collision dissociation of the triple protonated modified peptide it was verified that the additional 79.97 Da is localized to the C-terminal serine residue of the peptidic linker (see FIG. 4). This was further confirmed by spiking a synthetic polypeptide and enzymatic dephosphorylation (FIGS. 5 and 6). By digesting with thermolysin and LC-MS/MS, the phosphorylation could be localized to glycine-serine linker I of fusion protein 1 specifically (see FIGS. 7 and 8).

In order to reduce the heterogeneity of the HC1-F fusion protein the post-translational modification is removed by replacing the terminal serine residue of the peptidic linker with a different residue, but not with proline or threonine, preferably with glycine or by simply deleting the residue with the proviso that the next residue, i.e. the first residue of the polypeptide fused to the peptidic linker is also no serine, threonine or proline residue.

### Fusion Protein 2—HC2-F:

Fusion protein 2 comprises one peptidic linker connecting the C-terminus of an antibody heavy chain with the shortened N-terminus of an antibody light chain. It has been found that HC2-F is expressed with O-glycosylation of the serine residue in the linker SLSLPGGGGSGGGGSGGGGSGGGGSIQM SEQ ID NO: 10 (cf FIGS. 9-13). FIG. 9 shows the O-xylosylation and O-glycosylation on HC2-F after reduction and analysis by UHR-QTOF-ESI-MS. In the extracted ion chromatogram in FIG. 10 obtained from HC2-F an O-glycan was identified in peptide SLSLSPGGGGGSGGGGSGGGGSGGGGSIQMTX13 (SEQ ID NO: 10) comprising the glycine serine linker. Using EThcD/HCD MS² the localization of GalNAc (+203 Da) and GalNAc-Gal-Neu5Ac (+656 Da) at the C-terminal serine residue of the GS-peptidic linker was confirmed (FIGS. 11 and 12). FIG. 13 shows for HC2-F the relative quantification of the O-glycans of the HC peptide fragment of SEQ ID NO: 10 relative to the sum of the unmodified and O-xylosylated peptide.

In order to reduce the heterogeneity of the HC2-F fusion protein the post-translational modification is removed by replacing the terminal serine residue of the peptidic linker with a different residue, but not with proline or threonine, preferably with glycine or by simply deleting the residue with the proviso that the next residue, i.e. the first residue of the polypeptide fused to the peptidic linker is also no serine, threonine or proline residue.

Fusion protein 3—LC3-F: Fusion protein 3 comprises one peptidic linker connecting an antibody light chain with an antibody Fc-region. It has been found that LC3-F is expressed with O-glycosylation of the threonine residue in the linker GGGSGGGGSGGGGSGGGGSGGGGTCPPCPAPEAAGGPSVFLFPPKPK SEQ ID NO: 11 (cf. FIGS. 14-16). FIG. 14B shows O-glycosylation on LC3-F produced in HEK cells after N-deglycosylation and analysis by UHR-QTOF-ESI-MS of the intact protein. The modification GalNAc-Gal-2NeuAc (+948 Da) quantifies to an amount of about 20%. FIG. 14C shows the intact LC3-F after N-deglycosylation, desialidation and analysis by UHR-QTOF-ESI-MS of the intact protein. FIG. 14D demonstrates that the O-glycosylation is localized to chain A of LC3-F following N-deglycosylation, desialidation, and analysis by UHR-QTOF-ESI-MS of the reduced Fusion protein 3.

FIG. 15 shows for LC3-F the deconvoluted mass spectrum of an endoprotease digest (derived from Akkermansia muciniphila; OpeRATOR; Genovis). FIG. 16 shows the XICs for LC3-F following desialidation and tryptic digests with (upper XIC) or without (lower XIC) OpeRATOR protease. The masses of the fragments correspond to O-glycosylation of the hinge region threonine residue. MS/MS of the desialidated tryptic peptide digested with OpeRATOR protease that localizes the O-glycosylation to the N-terminal threonine residue (FIG. 17).

In order to reduce the heterogeneity of the LC3-F fusion protein, the post-translational modification is removed by replacing the first residue of the polypeptide fused to the peptidic linker, i.e. the threonine residue, but not with proline or serine, preferably with glycine.

In a variant of LC3-F the threonine residue at the end of the linker has been omitted and substituted by a glycine residue so that the linker comprises the amino acid sequence GGGGSGGGGSGGGGSGGGGSGGGGSGGGGGCPPC SEQ ID NO: 23. FIG. 21 shows the variant of LC3-F after N-deglycosylation, and analysis by UHR-QTOF-ESI-MS of the intact protein. FIG. 22 shows the variant of LC3-F after N-deglycosylation, reduction, and analysis by UHR-QTOF-ESI-MS. No O-glycosylation is present in the variant of LC3-F.

### Fusion Protein 4—HC4-F:

Fusion protein 4 comprises an peptidic linker connecting a non-IgG protein with an antibody Fc-region. It has been found that HC4-F is expressed with O-fucosylation of the serine residue in the linker LGGGGSGGGGSRT SEQ ID NO: 14 (cf. FIG. 18-19). FIG. 18 shows for HC4-F following a thermolysin digest that an O-fucosylation (+146 Da) is present and can be localized to the peptide LGGGGSGGGGSRT (SEQ ID NO: 14) by peptide mapping (XICs). FIG. 19 shows for HC4-F the results of spiking of modified synthetic peptide (27-310 nM) X8LGGGGSGGGGS(+Fucose)RT (SEQ ID NO: 15) into a tryptic digest. The modified synthetic peptide co-elutes with the modified HC4-F tryptic peptide X8LGGGGSGGGGSRT+146 Da and increases the area under the curve. Shown are the XICs without spiking (top) and increasing levels of spiking (below). LC-MS/MS tryptic peptide mapping localized the O-fucosylation to the terminal serine residue (FIG. 20). In order to reduce the heterogeneity of the HC4-F fusion protein, the post-translational modification is removed by replacing the terminal serine residue of the peptidic linker with a different residue, but not with proline or threonine, preferably with glycine or by simply deleting the residue with the proviso that the next residue, i.e. the first residue of the polypeptide fused to the peptidic linker is also no serine, threonine or proline residue.

### SPECIFIC EMBODIMENTS

1. A fusion polypeptide comprising the amino acid sequence

(SEQ ID NO: 04)
GyX1

wherein X1 can be any amino acid residue except for serine, threonine and proline, and
wherein y is an integer from and including 3 to 25.
2. The fusion polypeptide according to embodiment 1, wherein the fusion polypeptide comprises the amino acid sequence

(SEQ ID NO: 05)
GyX1X2X3

wherein X1 can be any amino acid residue except for serine, threonine and proline,
wherein X2 and X3 can be independently of each other any amino acid residue, and
wherein y is an integer from and including 3 to 25.
3. The fusion polypeptide according to any one of embodiments 1 to 2, wherein y is an integer from and including 4 to 20.
4. The fusion polypeptide according to any one of embodiments 1 to 3, wherein y is an integer from and including 5 to 15.
5. A fusion polypeptide comprising the amino acid sequence

(SEQ ID NO: 02)
GnSGmX1X2X3

wherein X1 can be any amino acid residue except for serine, threonine and proline,
wherein X2 and X3 can be independently of each other any amino acid residue,
wherein n=1, 2, 3 or 4, and
wherein m=3, 4 or 5.
6. The fusion polypeptide according to any one of embodiments 1 to 5, wherein the fusion polypeptide comprises at least three domains
   wherein each of the three domains is independently of the other two a polypeptide of at least 10 amino acid residues in length, and wherein the domains are conjugated to each other via peptide bonds.
7. The fusion polypeptide according to embodiment 6, wherein the C-terminus of the first domain is conjugated to the N-terminus of the second domain via a peptide bond, and the C-terminus of the second domain is conjugated to the N-terminus of the third domain via a peptide bond.
8. The fusion polypeptide according to any one of embodiments 1 to 7, wherein the fusion polypeptide is a recombinant fusion polypeptide.
9. The fusion polypeptide according to any one of embodiments 1 to 8, wherein the fusion polypeptide is produced in a eukaryotic cell.
10. The fusion polypeptide according to any one of embodiments 1 to 9, wherein the fusion polypeptide has no post-translational modification at S or X1 of SEQ ID NO: 01, or SEQ ID NO: 02, or SEQ ID NO: 04, respectively.
11. The fusion polypeptide according to any one of embodiments 1 to 10, wherein the linear fusion polypeptide has no post-translational modification at X1 of SEQ ID NO: 01, or SEQ ID NO: 02, or SEQ ID NO: 04.
12. The fusion polypeptide according to any one of embodiments 10 to 11, wherein the post-translational modification is phosphorylation (addition of a phosphate group) and/or glycosylation (addition of a carbohydrate moiety).
13. The fusion polypeptide according to embodiment 12, wherein the glycosylation is xylosylation (the carbohydrate moiety is xylose).
14. The fusion polypeptide according to embodiment 12, wherein the glycosylation is glucosylation (the carbohydrate moiety is glucose).
15. The fusion polypeptide according to any one of embodiments 5 to 14, wherein the fusion polypeptide has reduced post-translational modification at residues S and/or X1 compared to a fusion polypeptide comprising

(SEQ ID NO: 06)
GpSGnSGmX1X2X3

wherein X1 is serine, threonine or proline,
wherein X2 and X3 can be independently of each other any amino acid residue,
wherein n=1, 2, 3 or 4,
wherein p=n or p=1, 2, 3 or 4, and
wherein m=3, 4 or 5.
16. The fusion polypeptide according to any one of embodiments 5 to 15, wherein n=3 and m=3 or 4.
17. The fusion polypeptide according to any one of embodiments 5 to 15, wherein n=3 and m=4.
18. The fusion polypeptide according to any one of embodiments 5 to 15, wherein n=4 and m=4 or 5.
19. The fusion polypeptide according to any one of embodiments 5 to 15, wherein n=4 and m=5.
20. The fusion polypeptide according to any one of embodiments 1 to 19, wherein the fusion polypeptide comprises the amino acid sequence

(SEQ ID NO: 03)
GpSGnSGmX1X2X3

wherein X1 can be any amino acid residue except for serine, threonine and proline,
wherein X2 and X3 can be independently of each other any amino acid residue,
wherein n=1, 2, 3 or 4,
wherein m=3, 4 or 5, and
wherein p=3 or 4.
21. The fusion polypeptide according to any one of embodiments 1 to 20, wherein the fusion polypeptide further comprises an antibody Fc-region polypeptide/at least one domain is an antibody Fc-region polypeptide.
22. The fusion polypeptide according to any one of embodiments 6 to 21, wherein the first or/and the third domain is an antibody Fc-region polypeptide.
23. The fusion polypeptide according to any one of embodiments 1 to 22, wherein the fusion polypeptide further comprises a VH domain, a VL domain, a scFv, a scFab, a VH-CH1 pair, a VL-CL pair, a VH-CL pair, a VL-CH1 pair, a receptor or extracellular domain thereof, a receptor binding portion of a ligand, an enzyme, a growth factor, an interleukin, a cytokine, or a chemokine/according to any one of embodiments 2 to 18, wherein at least one domain of the fusion polypeptide is selected from the group consisting of a VH domain, a VL domain, as scFv, a scFab, a VH-CH1 pair, a VL-CL pair, a VH-CL pair, a VL-CH1 pair, a receptor or extracellular domain thereof, a receptor binding portion of a ligand, an enzyme, a growth factor, an interleukin, a cytokine, and a chemokine.
24. The fusion polypeptide according to any one of embodiments 1 to 23, wherein the fusion polypeptide is monomeric.
25. The fusion polypeptide according to any one of embodiments 1 to 24, wherein the fusion polypeptide is a linear fusion polypeptide.
26. A multimeric molecule comprising at least two polypeptides whereof at least one is a fusion polypeptide according to any one of embodiments 1 to 25.
27. The multimeric molecule according to embodiment 26, wherein the at least two polypeptides are conjugated to each other by one or more disulfide bonds.
28. The multimeric molecule according to any one of embodiments 26 to 27, wherein the multimeric molecule is an antibody.
29. The multimeric molecule according to any one of embodiments 26 to 28, wherein the multimeric molecule is a bispecific antibody.
30. The multimeric molecule according to any one of embodiments 26 to 29 further comprising at least one antibody light chain.
31. The multimeric molecule according to any one of embodiments 26 to 30 further comprising at least one antibody heavy chain.
32. A pharmaceutical composition comprising at least one fusion polypeptide according to any one of embodiments 1 to 25 or a multimeric molecule according to any one of embodiments 26 to 31 and optionally a pharmaceutically acceptable carrier.
33. A nucleic acid molecule encoding a fusion polypeptide according to any one of embodiments 1 to 26.
34. The nucleic acid molecule according to embodiment 33, wherein the nucleic acid molecule is in an expression cassette.
35. The nucleic acid molecule according to any one of embodiments 34 to 35, wherein the nucleic acid molecule is in a vector.
36. A set of nucleic acid molecules encoding the polypeptides of the multimeric molecule according to any one of embodiments 26 to 31.
37. The set of nucleic acid molecules according to embodiment 36, wherein each of the nucleic acid molecules is in an expression cassette.
38. The set of nucleic acid molecules according to any one of embodiments embodiment 36 to 37, wherein each of the nucleic acid molecules is in a vector.
39. The set of nucleic acid molecules according to any one of embodiment 36 to 37, wherein the nucleic acid molecules are on the same vector.
40. The set of nucleic acid molecules according to any one of embodiment 36 to 37, wherein the nucleic acid molecules are on different vectors.
41. The set of nucleic acid molecules according to any one of embodiment 36 to 37, wherein the nucleic acid molecules are on two vectors.
42. A eukaryotic cell comprising the nucleic acid molecule according to embodiment 33 or the set of nucleic acid molecules according to embodiment 36.
43. A peptidic linker comprising the amino acid sequence

(SEQ ID NO: 04)
GyX1

wherein X1 can be any amino acid residue except for serine, threonine and proline, and
wherein y is an integer from and including 3 to 25.
44. A peptidic linker comprising the amino acid sequence

(SEQ ID NO: 05)
GyX1X2X3

wherein X1 can be any amino acid residue except for serine, threonine and proline,
wherein X2 and X3 can be independently of each other any amino acid residue, and
wherein y is an integer from and including 3 to 25.
45. The peptidic linker according to any one of embodiments 43 to 44, wherein y is an integer from and including 4 to 20.
46. The peptidic linker according to any one of embodiments 43 to 45, wherein y is an integer from and including 5 to 15.
47. A peptidic linker comprising the amino acid sequence

(SEQ ID NO: 01)
GnSGmX1X2X3

wherein X1 can be any amino acid residue except for serine, threonine and proline,
wherein X2 and X3 can be independently of each other any amino acid residue,
wherein n=1, 2, 3 or 4, and
wherein m=3, 4 or 5.
48. A peptidic linker comprising the amino acid sequence

(SEQ ID NO: 03)
GpSGnSGmX1X2X3

wherein X1 can be any amino acid residue except for serine, threonine and proline,
wherein X2 and X3 can be independently of each other any amino acid residue,
wherein n=1, 2, 3 or 4,
wherein m=3, 4 or 5, and
wherein p=3 or 4.
49. The fusion polypeptide according to any one of embodiments 47 to 48, wherein n=3 and m=3 or 4.
50. The fusion polypeptide according to any one of embodiments 47 to 49, wherein n=3 and m=4.
51. The fusion polypeptide according to any one of embodiments 47 to 48 wherein n =4 and m =4 or 5.
52. The fusion polypeptide according to any one of embodiments 47 to 48 and 51, wherein n=4 and m=5.
53. A method for producing a fusion polypeptide comprising the following steps:
   cultivating the eukaryotic cell according to embodiment 42 under conditions where the fusion polypeptide is expressed, and
   recovering the fusion polypeptide from the eukaryotic cell or the cultivation medium.

thereby producing a fusion polypeptide.
54. A method of producing a fusion polypeptide having reduced levels of post-translational modification comprising:
   cultivating the host cell according to embodiment 42 under conditions where the fusion polypeptide is expressed, and
   recovering the fusion polypeptide from the eukaryotic cell or the cultivation medium,
   thereby producing a fusion polypeptide having reduced levels of post-translational modifications.
55. A method of producing a stabilized fusion polypeptide comprising genetically engineering a fusion protein to comprise a peptidic linker of any one of embodiments 43 to 52 and causing the fusion polypeptide to be expressed by a eukaryotic cell, and thereby producing a stabilized fusion polypeptide.
56. A composition made by the method according to any one of embodiments 53 to 55.
57. A method of treating a subject that would benefit from treatment with a fusion polypeptide according to any one of embodiments 1 to 25 or a multimeric molecule according to any one of embodiments 26 to 31, comprising administering a composition of anyone of embodiments 32 or 56 to the subject.
58. Use of a composition according to any one of embodiments 32 or 56 for the treatment of a disease or disorder.
59. Use of a composition according to any one of embodiments 32 or 56 for the manufacture of a medicament.
60. The fusion polypeptide according to any one of embodiments 1 to 25 or the multimeric molecule according to any one of embodiments 26 to 31 for use as a medicament.
61. Use of the fusion polypeptide according to any one of embodiments 1 to 25 or the multimeric molecule according to any one of embodiments 26 to 31 in the manufacture of a medicament.
62. A method of treating an individual in need of a treatment comprising administering to the individual an effective amount of the fusion polypeptide according to any one of embodiments 1 to 25 or the multimeric molecule according to any one of embodiments 26 to 31.

### DESCRIPTION OF THE FIGURES

FIG. 1 shows the total mass determination of a fusion protein comprising an antibody heavy chain constant domain and non-antibody polypeptides (HC1-F) transiently expressed in human embryonic kidney cells. A deconvoluted mass spectrum of the deglycosylated and reduced HC1-F is shown. In addition to the expected HC1-F, another peak evidencing the presence of an intense +79 Da product variant of HC1-F can be seen. A less intense signal due to a +132 Da xylose product variant was also present.

FIG. 2 depicts a schematic representation of HC1-F. HC1-F consists of two >10 kDa non-IgG proteins fused by two standard glycine-serine [(G4S)2] linkers I and II to an IgG heavy chain constant domain.

FIG. 3 shows the extracted ion current (EIC) chromatograms (z=2 and 3) of (A) the unmodified (elution time 36.5 min) and (B) the +79.97 Da modified (elution time 37.6 min) tryptic digested glycine-serine linker peptides (X9GGGGSGGGGSR; SEQ ID NO: 07) of HC1-F. A relative comparison of the integrated EIC chromatograms quantified the modification to amount to 5.5% (including 1.1% with O-xylose). MA, manually integrated peak; NL, normalized intensity level.

FIG. 4 shows the ion trap MS/MS data obtained by collision induced dissociation of the triple protonated (A) unmodified and (B)+79.97 Da modified tryptic digested glycine-serine linker peptide of HC1-F, and (C) MS/MS spectrum by electron-transfer/higher-energy collision dissociation of the triple protonated modified peptide. The additional 79.97 Da is localized to the C-terminal serine residue.

FIG. 5 shows the effect of spiking of a synthetic phosphopeptide to the tryptic digest. Extracted ion current chromatograms (z=2 and 3) of the unmodified and the +79.97 Da modified tryptic digested linker peptide (X9GGGGSGGGGSR; SEQ ID NO: 07) of HC1-F (A) without, (B) with 0.5 μM, and (C) with 1.0 μM spiked synthetic phosphopeptide (X9GGGGSGGGGpSR; SEQ ID NO: 08). The spiked samples demonstrate increased peak area for the phosphorylated peptide, supporting the correct identification of the modified tryptic peptide. NL, normalized intensity level.

FIG. 6 shows the results of the enzymatic dephosphorylation of the modified tryptic digested linker peptide of HC1-F with alkaline phosphatase. Extracted ion current chromatograms (z=2 and 3) of the unmodified and the +79.97 Da modified tryptic linker peptide (X9GGGGSGGGGSR; SEQ ID NO: 07) incubated (A) with and (B) without alkaline phosphatase (control reaction). The modified tryptic peptide was no longer detectable following the enzymatic dephosphorylation. NL, normalized intensity level.

FIG. 7 shows that the specific phosphorylation is of the glycine-serine linker I. (A) Extracted ion current chromatogram (EIC) (z=2) of the unmodified (elution time 12.5 min) and the phosphorylated (elution time 13.5 min) thermolysin digested glycine-serine linker I peptide (XGGGGSGGGGSREX3; SEQ ID NO: 09) of HC1-F. A relative comparison of the integrated EIC chromatograms quantified the modification to amount for 11.3% (including 1.4% with O-xylose at the GSG motif (data not shown)). Orbitrap HCD-MS/MS data of the double protonated unmodified and modified thermolysin digested glycine-serine linker I peptide is shown in (B) and (C), respectively. The +79.97 Da modification is localized to the C-terminal serine residue. MA, manually integrated peak; NL, normalized intensity level.

FIG. 8 shows linker phosphorylation in HC1-F stably expressed in Chinese hamster ovary cells. (A) Extracted ion current (EIC) chromatograms (z=2) of the unmodified (elution time 15.6 min) and the phosphorylated (elution time 16.85 min) thermolysin digest glycine-serine linker I peptide (XGGGGSGGGGSREX3; SEQ ID NO: 09). The phosphorylated peptide was quantified to amount for 0.4%. (B) HCD-MS/MS data of the double protonated modified thermolysin digest peptide. MA, manually integrated peak; NL, normalized intensity level.

FIG. 9 shows the O-xylosylation and O-glycosylation on fusion protein 2 (produced in CHO cells; HC2-F) after reduction and analysis by UHR-QTOF-ESI-MS.

FIG. 10 was obtained using HC2-F: Main signal induced by Fc-N-Glycan (RT 16 min, 62 to 66 min). O-glycan identified (RT 43 to 46 min) for peptide SLSLSPGGGGGSGGGGSGGGGSGGGGSIQMTX13 (SEQ ID NO: 10) comprising a glycine serine linker.

FIG. 11 shows for HC2-F an MS/MS of peptide fragment comprising O-glycosylation of FIG. 10 with O-glycan and peptide identification.

FIG. 12 shows for HC2-F the results of an MS/MS for the peptide of SEQ ID NO: 10. Using EThcD/HCD MS² allowed the localization of GalNAc (+203 Da) and GalNAc-Gal-NeuSAc (+656 Da) to the C-terminal serine residue of the GS-peptidic linker. Besides that, several O-xylose hits (+132 Da) could be localized to other serine residues in the peptidic linker.

FIG. 13 shows for HC2-F the relative quantification of the O-glycans of the HC peptide fragment of SEQ ID NO: 10 relative to the sum of the unmodified and O-xylosylated peptide.

FIG. 14A shows O-glycosylation on fusion protein 3 (LC3-F; produced in HEK cells) after analysis by UHR-QTOF-ESI-MS of the intact protein. FIG. 14B shows deconvoluted mass spectra of the N-deglycosylated intact protein comprising LC3-F produced in HEK cells, FIG. 14C shows deconvoluted mass spectra of the N-deglycosylated and desialidated intact protein comprising LC-3F, and FIG. 14D shows deconvoluted mass spectra of N-deglycosylation and reduction of LC3-F. The determined molecular masses for the annotated variants for the N-deglycosylated intact protein or LC-3F are listed (Annotations: B: +364.8 Da (GalNAc-Gal), +656.8 Da (GalNAc-Gal-Neu5Ac), +948.2 Da (GalNAc-Gal-2Neu5Ac), +1313.1 Da (2xGalNAc-Gal-Neu5Ac), +1602.2 Da (GalNAc-Gal-Neu5Ac and GalNAc-Gal-2Neu5Ac), +1896.7 (2xGalNAc-Gal-2Neu5Ac). C: +365.7 Da (GalNAc-Gal), +731.0 Da (2xGalNAc-Gal) and chain A (C: +365.5 Da (GalNAc-Gal), +729.8 Da (2xGalNAc-Gal)). GalNAc (square), Gal (circle), and Neu5Ac (rhomb). The modification GalNAc-Gal-2NeuAc (+948 Da) quantifies to an amount of about 20%.

FIG. 15 shows for LC3-F the deconvoluted mass spectrum of the OpeRATOR digest. The masses of the fragments correspond to O-glycosylation of the hinge region threonine residue. OpeRATOR is derived from Akkermansia muciniphila and expressed in E. coli. The enzyme contains a His-tag and the molecular weight is 42 kDa.

FIG. 16 shows the XICs for LC3-F following desialidation and tryptic digests with (upper XIC) or without (lower XIC) OpeRATOR protease. It can be seen that the O-glycosylation (+365.13 Da=GalNAc-Gal) is localized to the threonine residue in the following sequence (underlined) GGGSGGGGSGGGGSGGGGSGGGG TCPPCPAPEAAGGPSVFLFPPKPK (SEQ ID NO: 11), i.e. C-terminal to a GS-peptidic linker (identified by peptide mapping (LC-MS/MS)). Shown are the XICs of the O-glycosylated tryptic peptide digested at the O-glycosylated threonine residue (T(+365.13)C(+59.01)PPC(+59.01)PAPEAAGGPSVFLFPPKP; SEQ ID NO: 12) and the unmodified tryptic peptide (XX(+59.01)GGGSGGGGSGGGGSGGGGSGGGGTC(+59.01) PPC(+59.01)PAPEAAGGPSVFLFPPKPK; SEQ ID NO: 13). +59.01=carboxymethylation.

FIG. 17 shows for LC3-F an MS/MS of the desialidated tryptic peptide digested with OpeRATOR protease that localizes the O-glycosylation (+365.13 Da=GalNAc-Gal) to the N-terminal threonine residue. +59.01=carboxymethylation.

FIG. 18 shows for fusion protein 4 (HC4-F) following a thermolysin digest that an O-fucosylation (+146 Da) is present and can be localized to the peptide LGGGGSGGGGSRT (SEQ ID NO: 14) by peptide mapping (LC-MS/MS). Shown are the XICs of the modified (lower XIC, 2.06%) and unmodified (upper XIC) thermolysin digest peptide fragments.

FIG. 19 shows for HC4-F the results of spiking of modified synthetic peptide (27-310 nM) X8LGGGGSGGGGS(+Fucose)RT (SEQ ID NO: 15) into a tryptic digest. The modified synthetic peptide co-elutes with the modified HC4-F tryptic peptide X8LGGGGSGGGGSRT+146 Da and increases the area under the curve. Shown are the XICs without spiking (top) and increasing levels of spiking (below).

FIG. 20 shows for HC4-F the MS/MS results of a tryptic peptide that localizes the O-fucosylation (+146 Da) to the C-terminal serine residue.

FIG. 21 shows for the variant of LC3-F the mass spectrum (upper full scale, lower zoom) after N-deglycosylation, and analysis by UHR-QTOF-ESI-MS of the intact protein. No O-glycosylation is present in the variant of LC3-F with a linker comprising the amino acid sequence GGGGSGGGGSGGGGSGGGGSGGGGSGGGGCPPC (SEQ ID NO: 23).

FIG. 22 shows for the variant of LC3-F the mass spectrum (upper full scale, lower zoom) after N-deglycosylation, and analysis by UHR-QTOF-ESI-MS of the reduced protein. No O-glycosylation is present in the variant of LC3-F with a linker comprising the amino acid sequence GGGGSGGGGSGGGGSGGGGSGGGGSGGGGGCPPC (SEQ ID NO: 23).

### EXAMPLES

### Sample Overview:

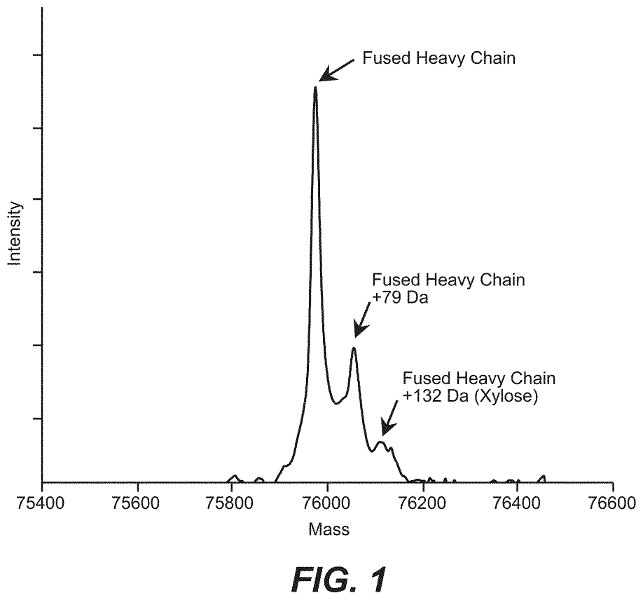

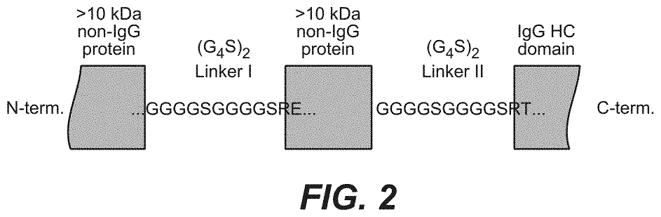

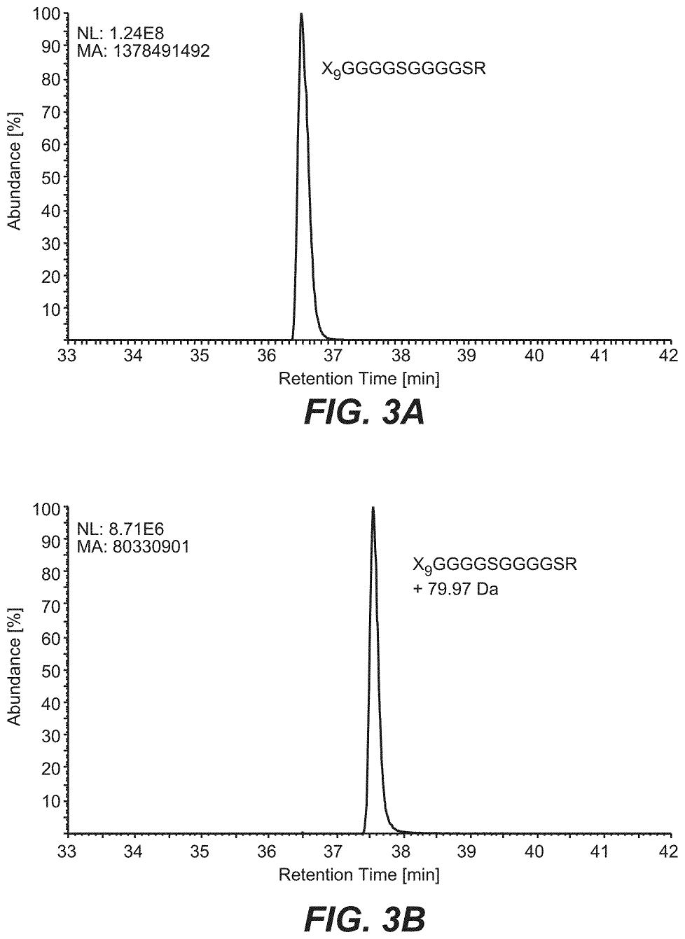

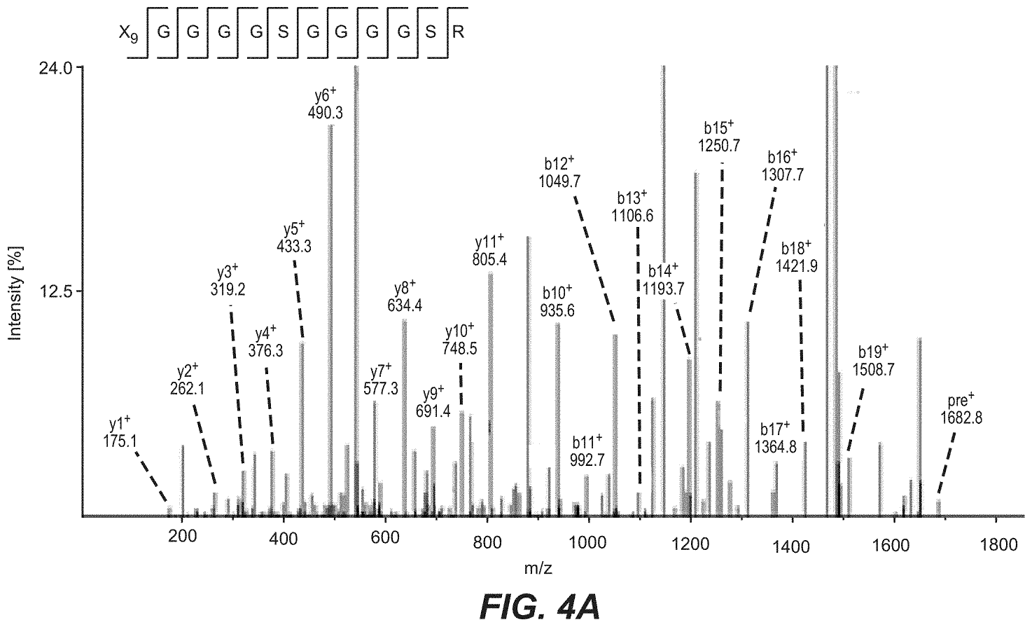

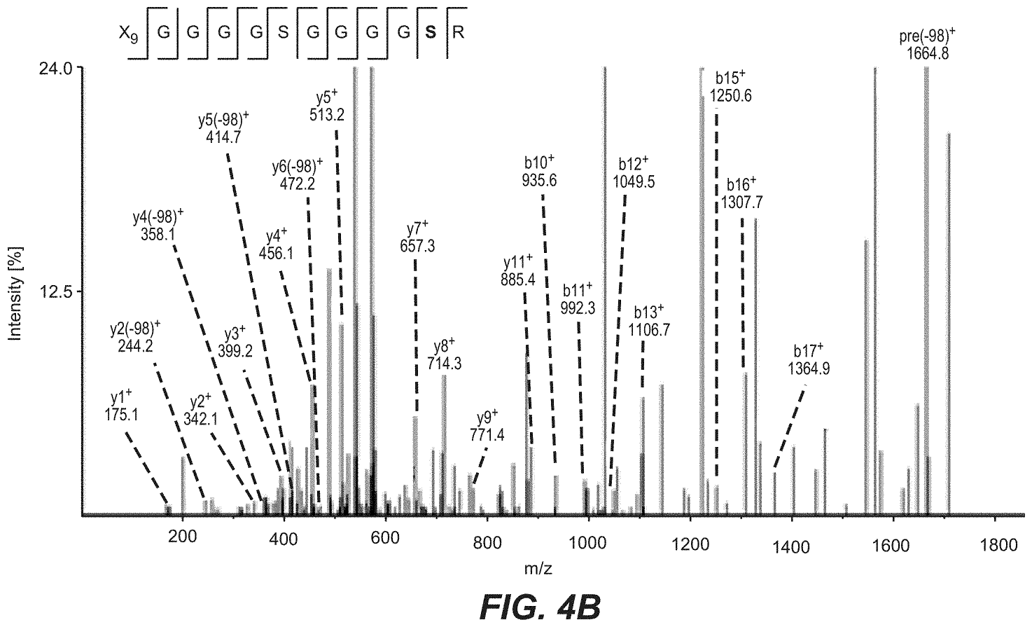

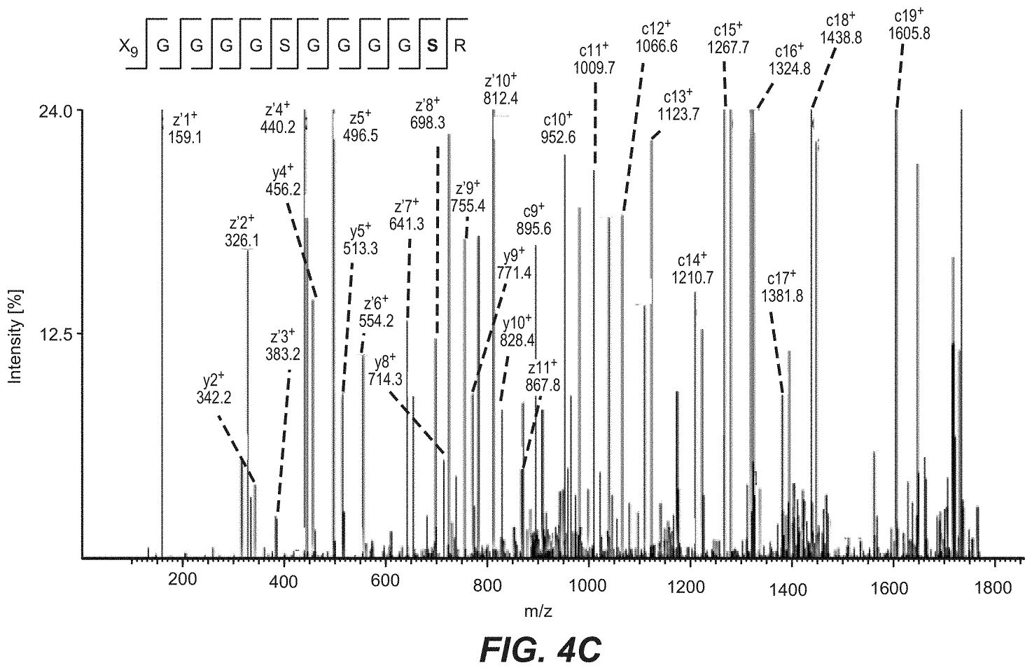

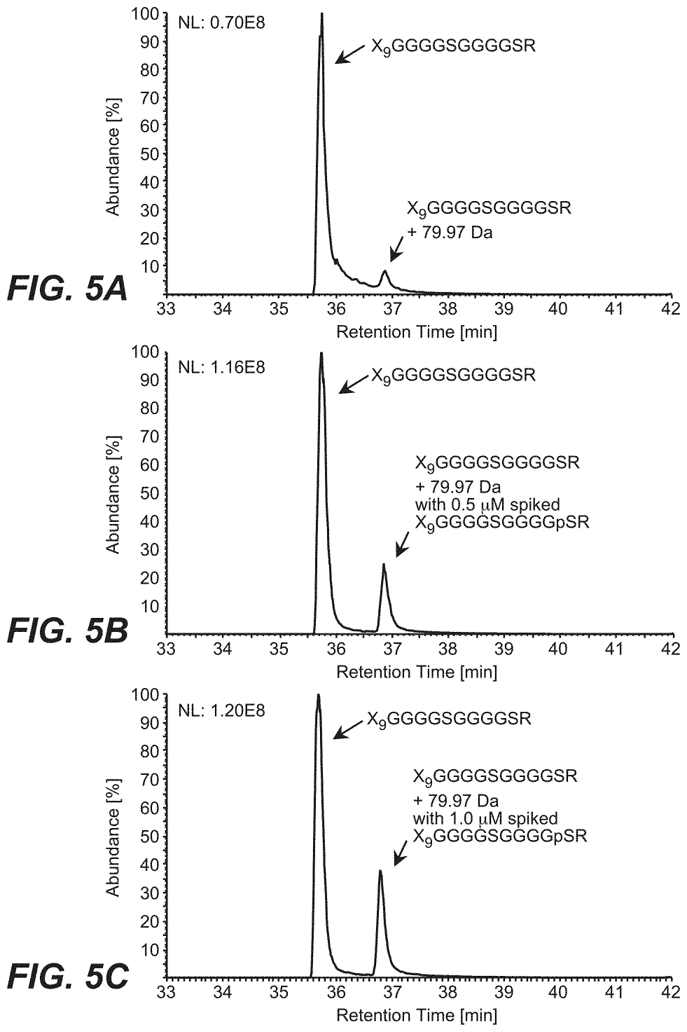

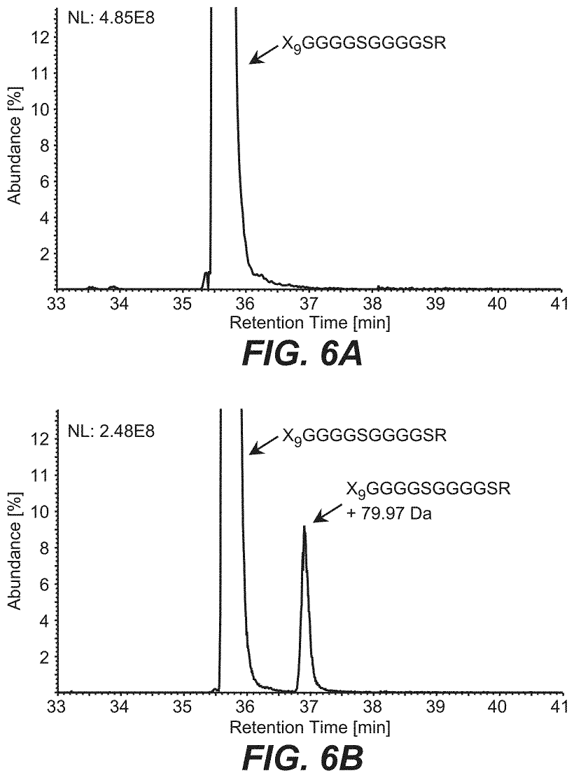

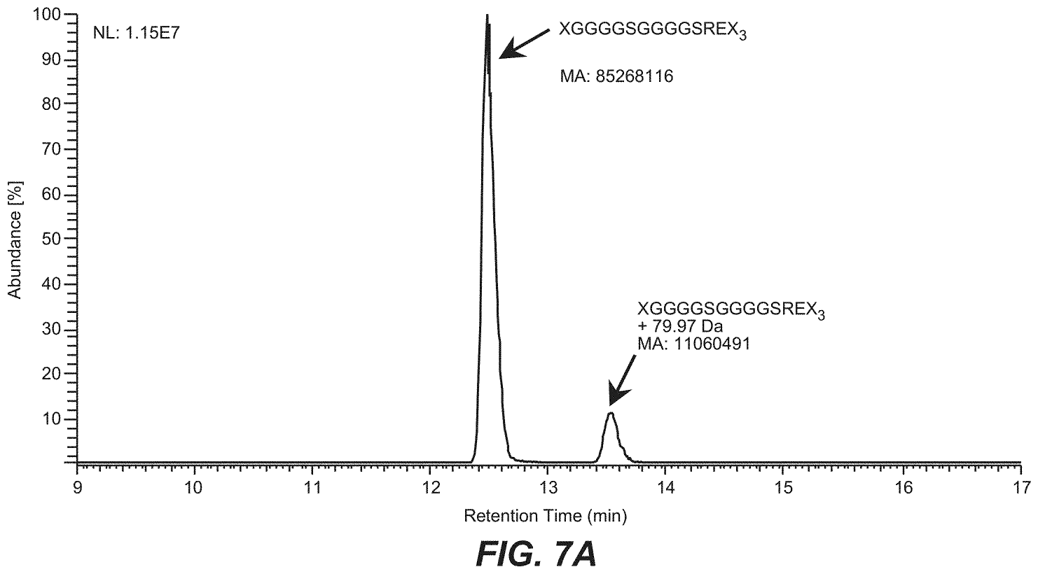

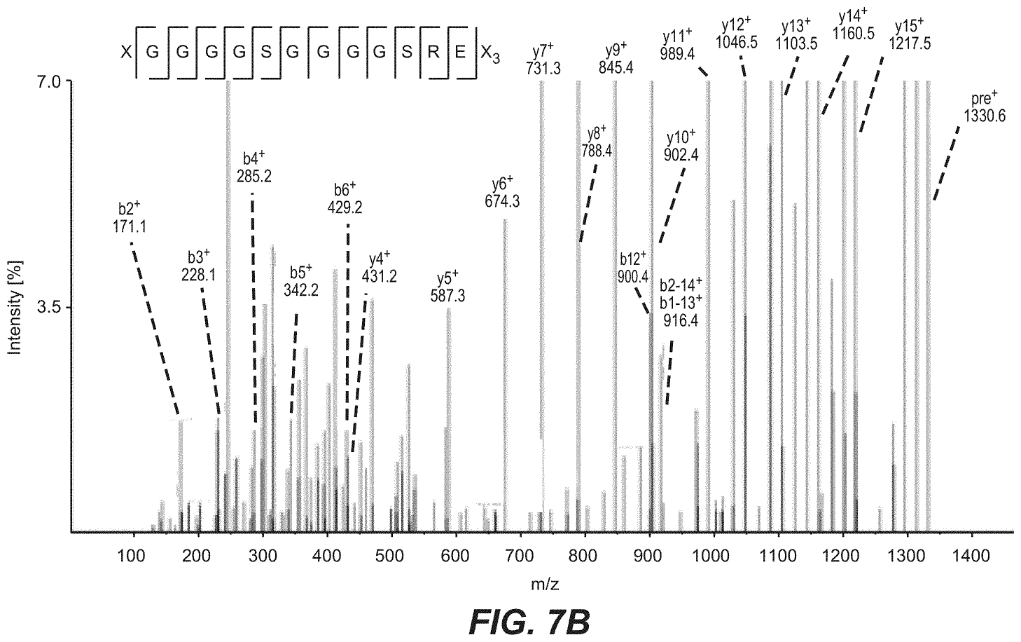

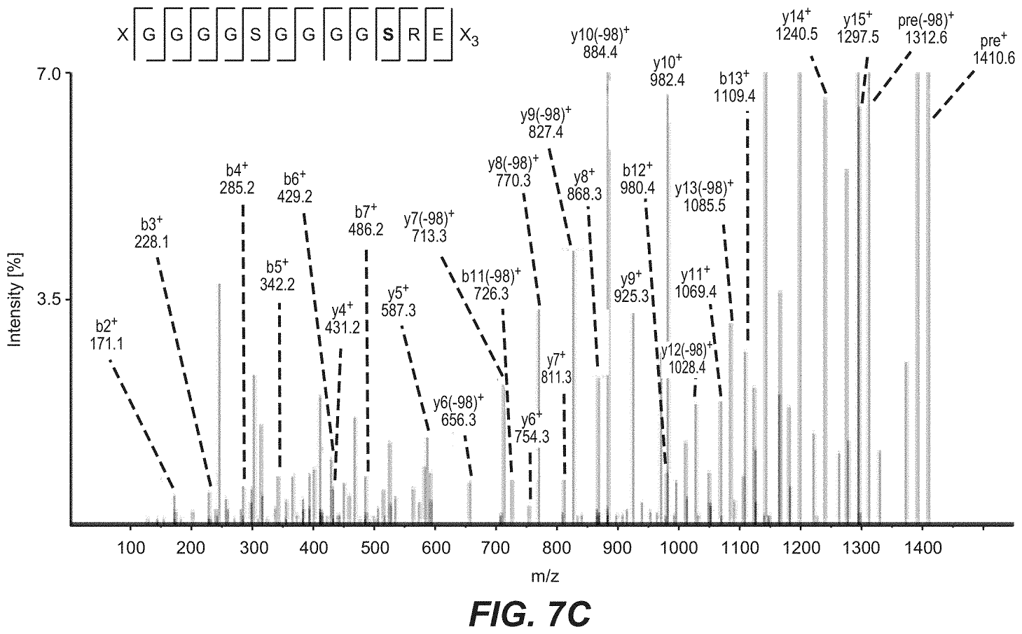

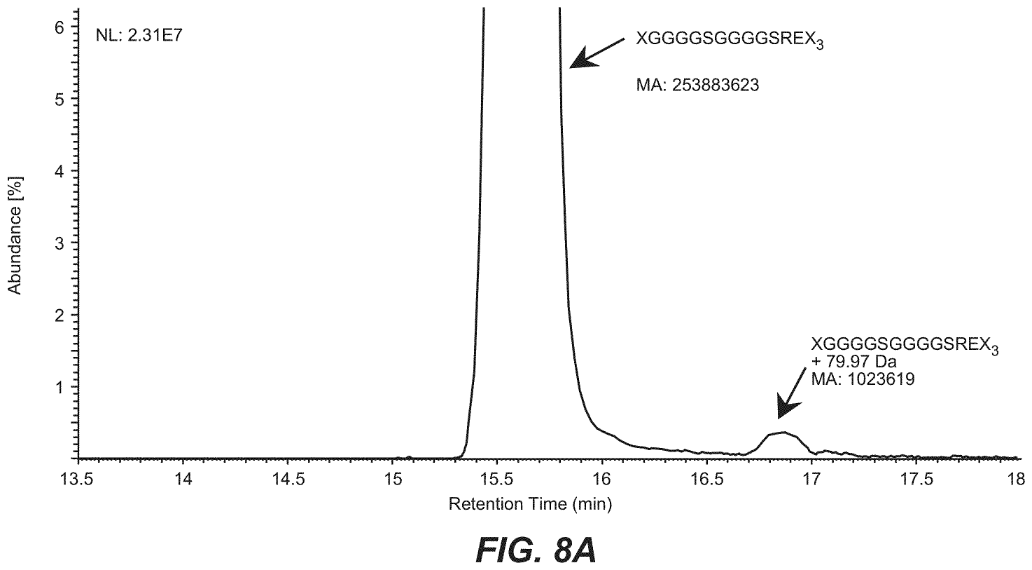

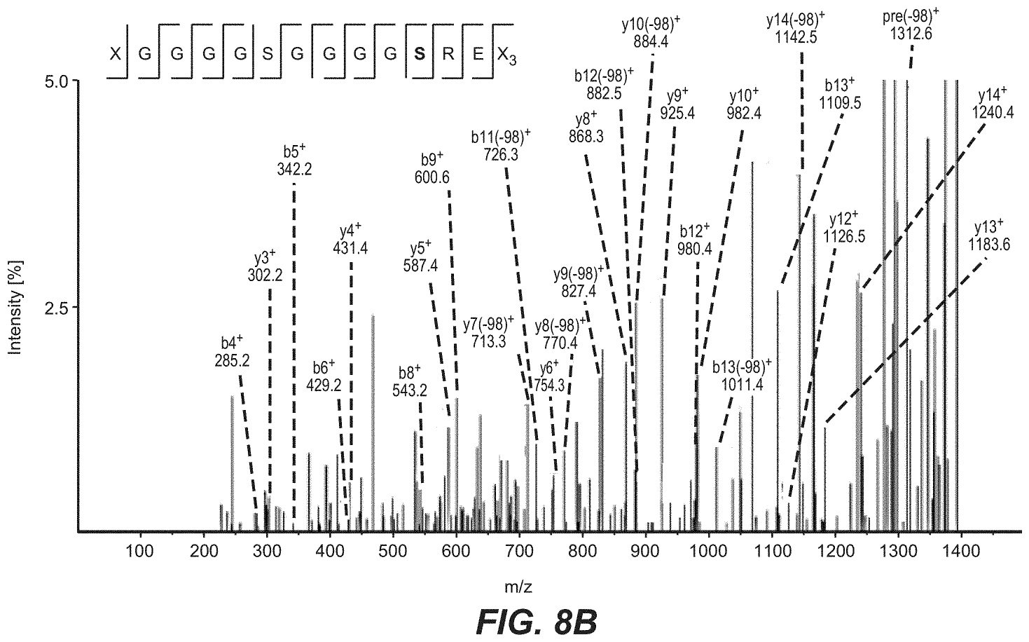

Fusion protein 1—HC1-F: with phosphorylation of the serine residue in the linker GGGGSGGGGSRE SEQ ID NO: 09 (cf FIGS. 1-8)

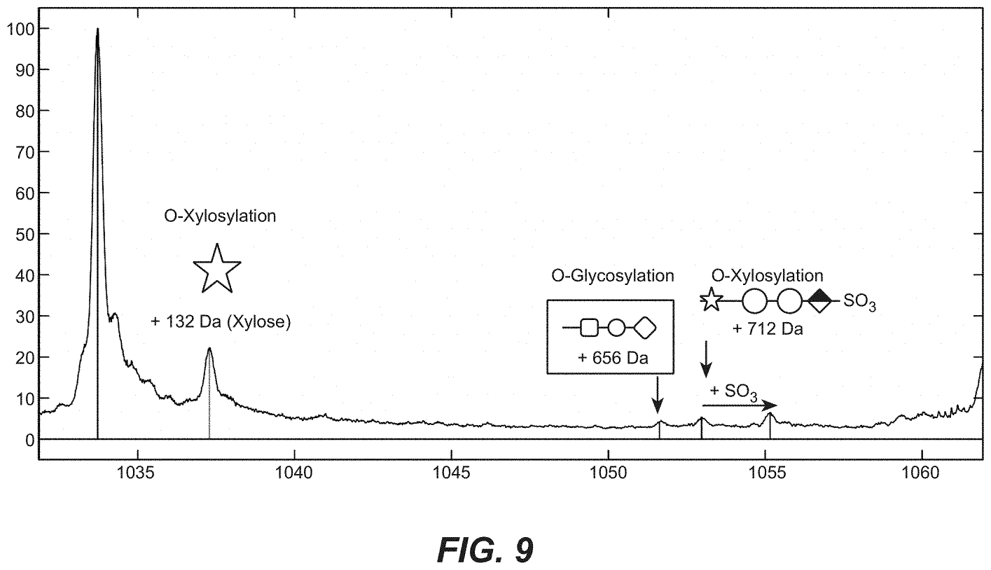

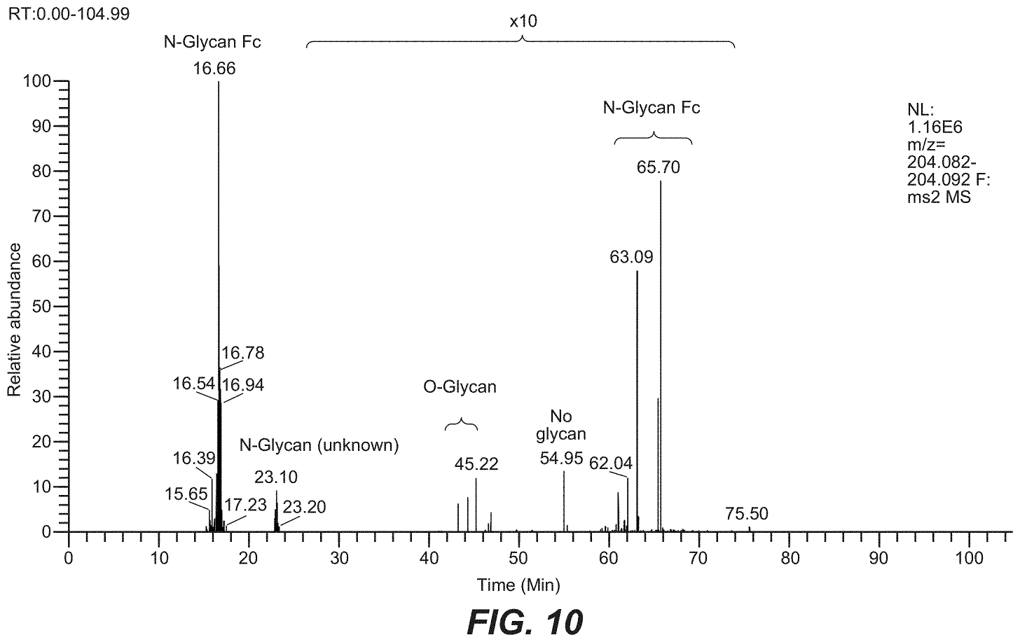

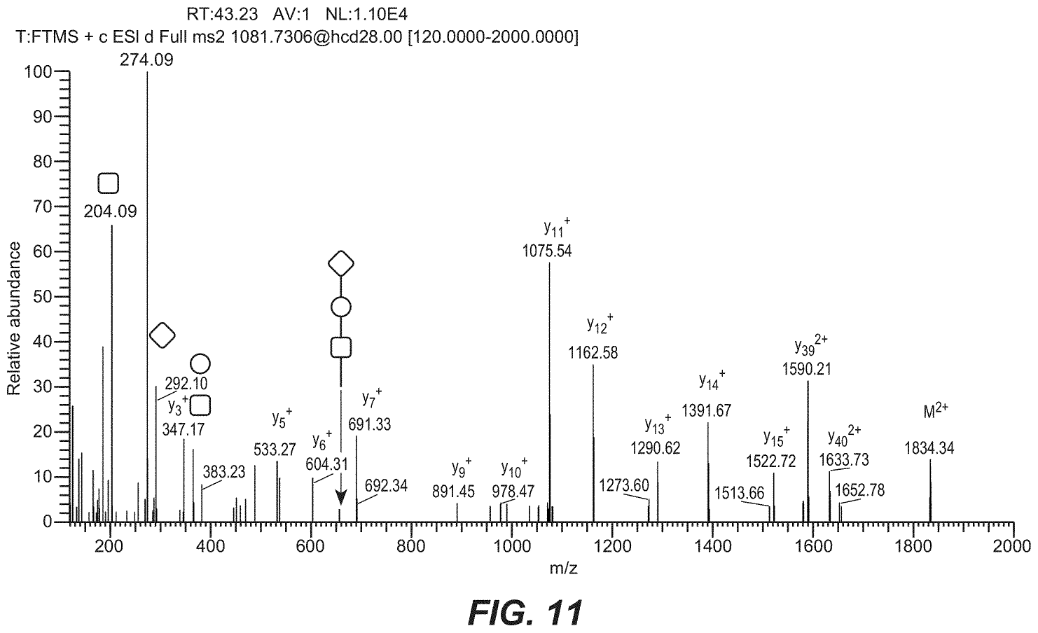

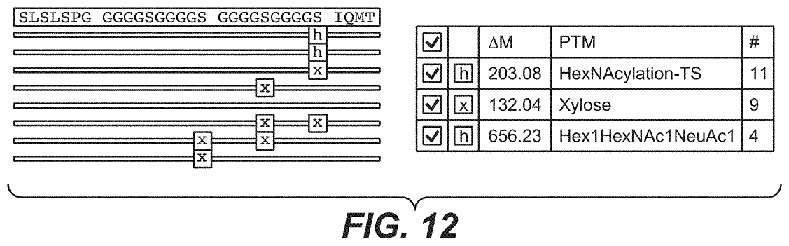

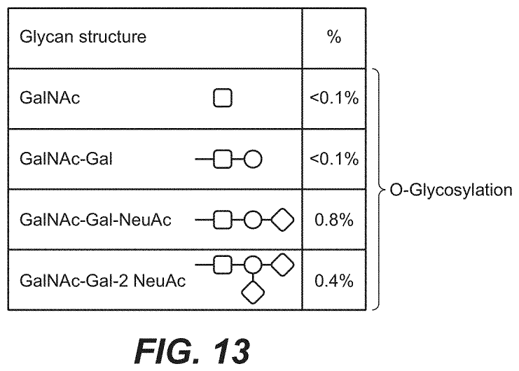

Fusion protein 2—HC2-F: with O-glycosylation of the serine residue in the linker SLSLPGGGGSGGGGSGGGGSGGGGSIQM SEQ ID NO: 10 (cf. FIGS. 9-13)

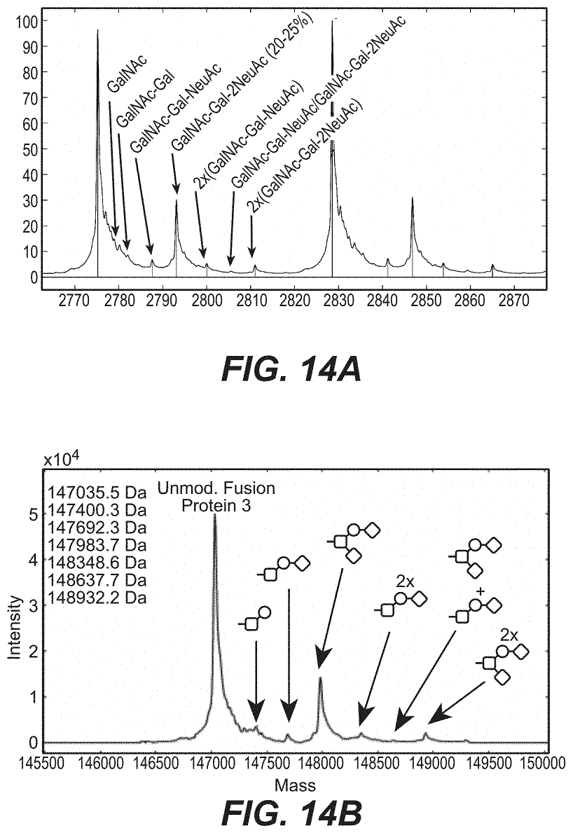

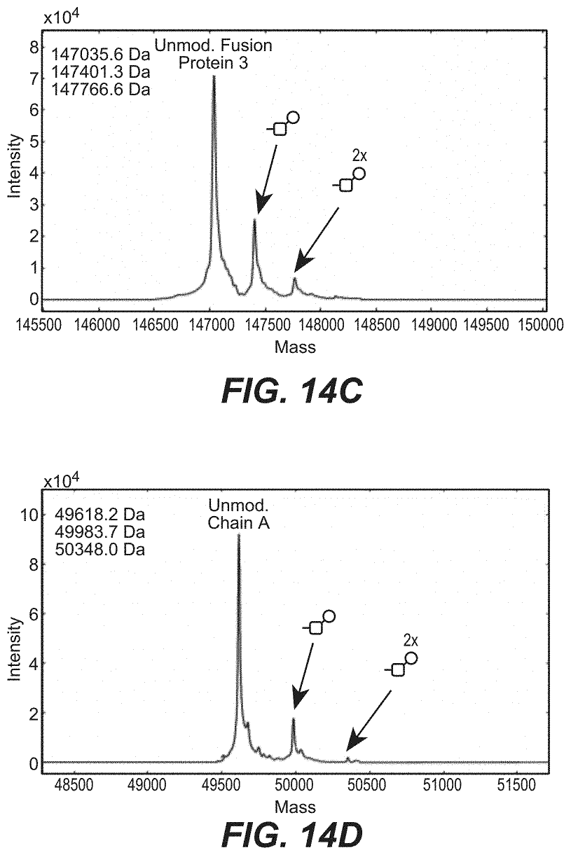

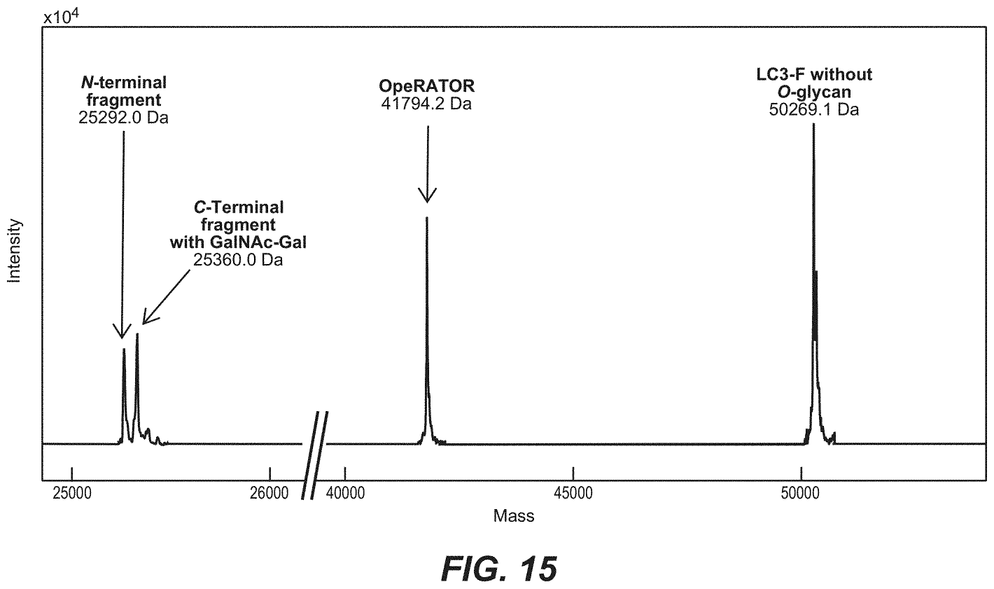

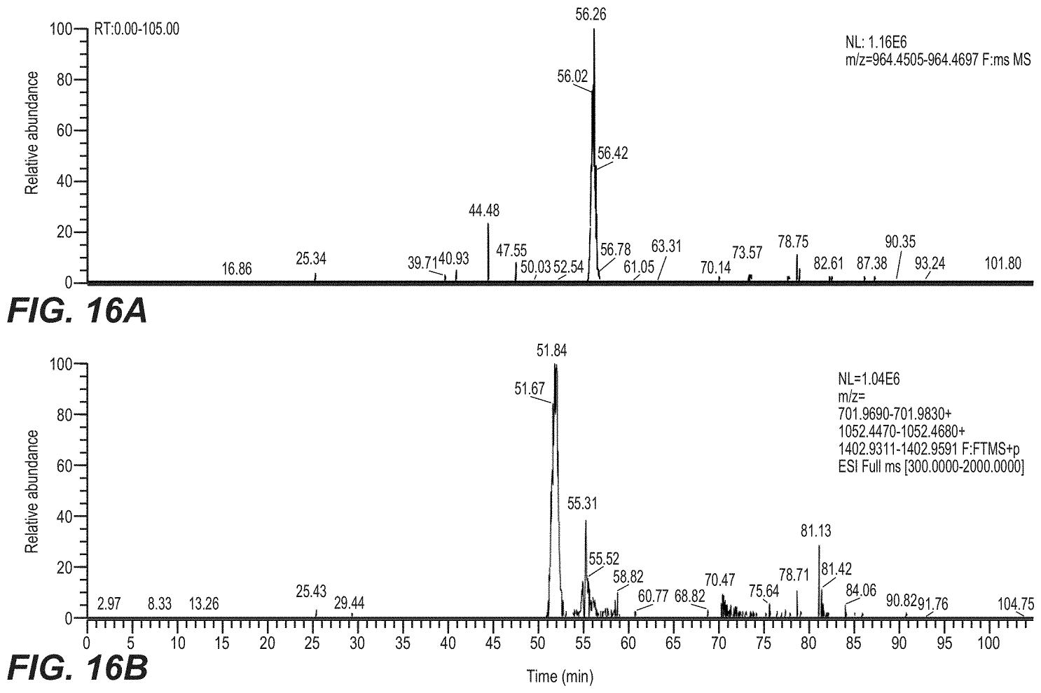

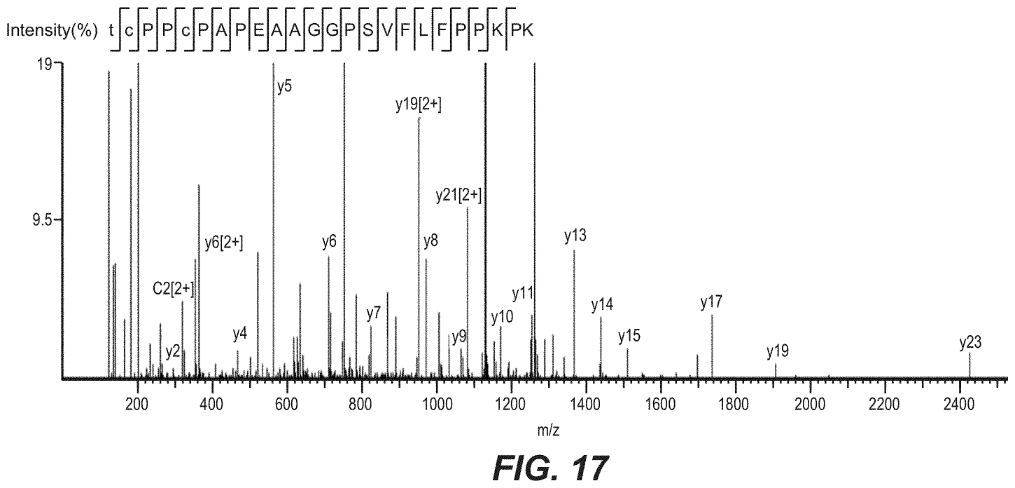

Fusion protein 3—LC3-F: with O-glycosylation of the threonine residue in the linker GGGSGGGGSGGGGSGGGGSGGGGTCPPCPAPEAAGGPSVFLFPPKPK SEQ ID NO: 11 (cf. FIGS. 14-17)

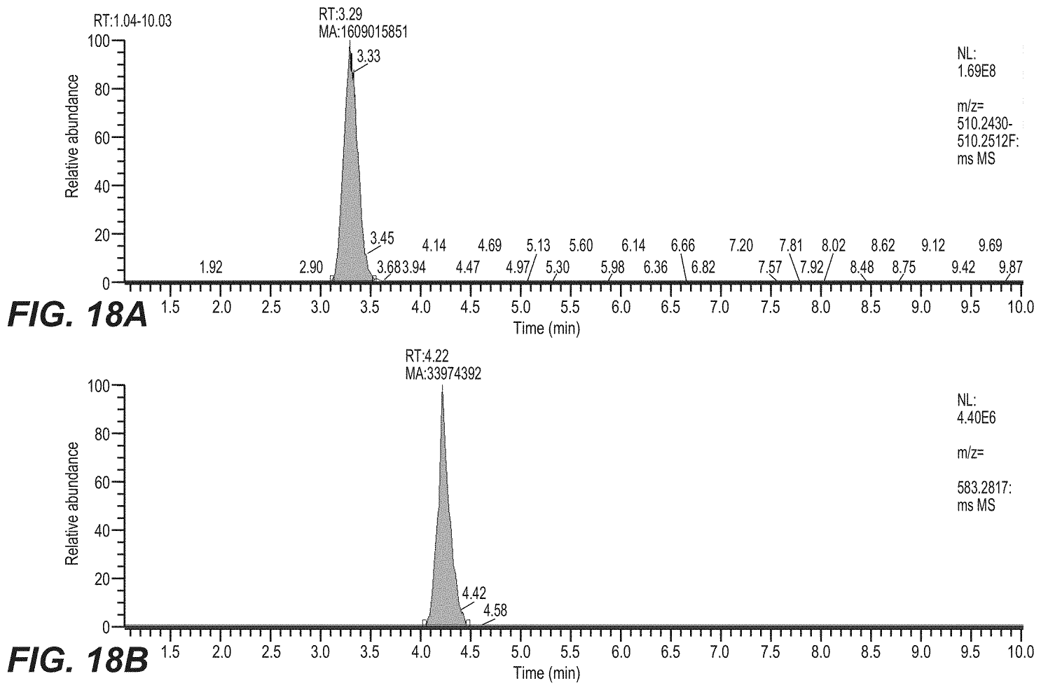

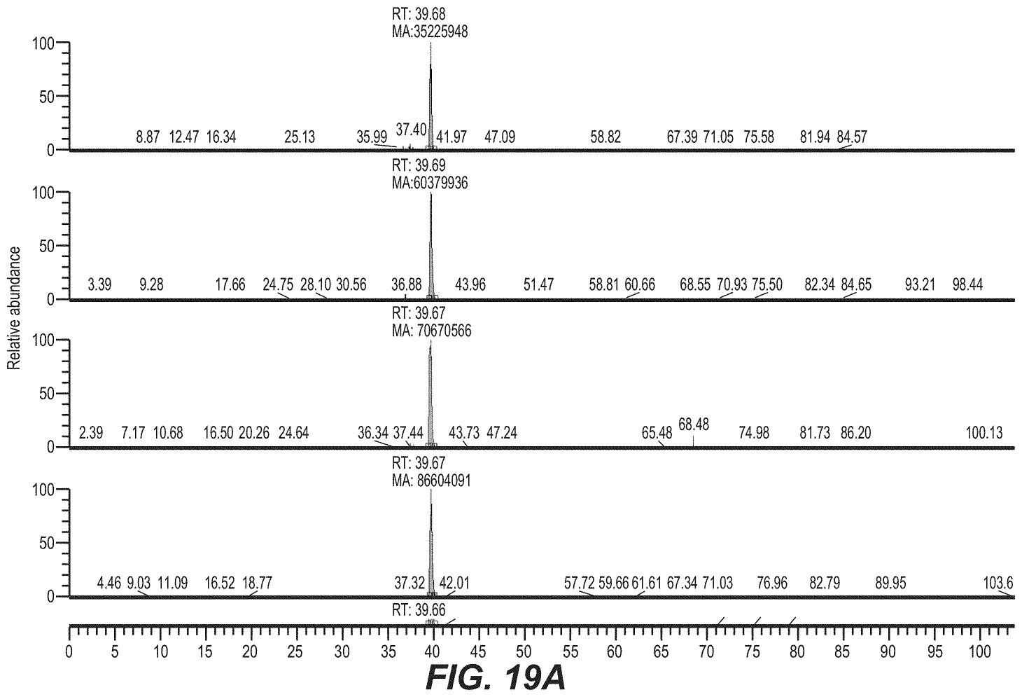

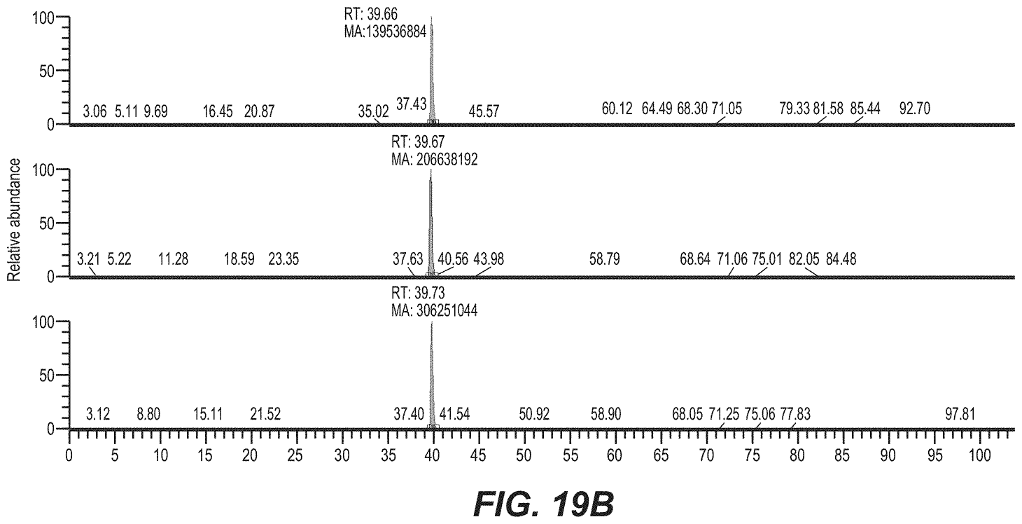

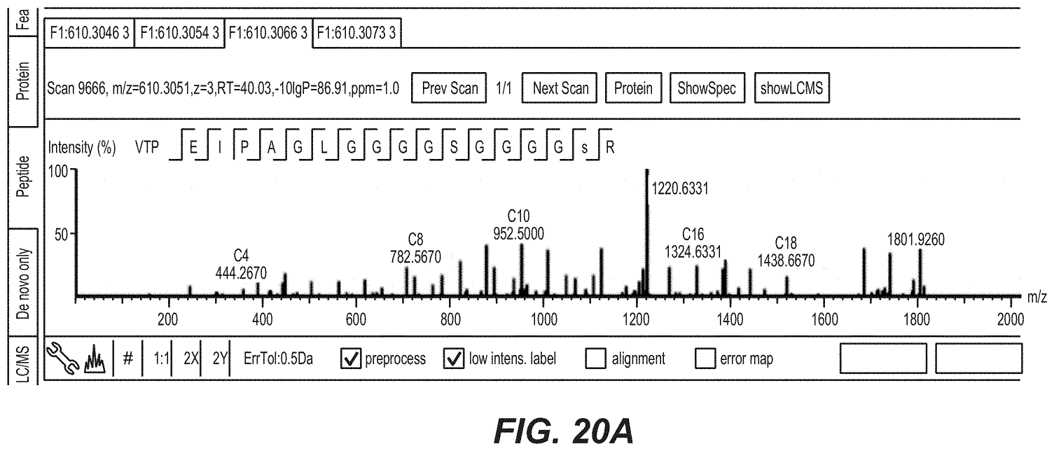

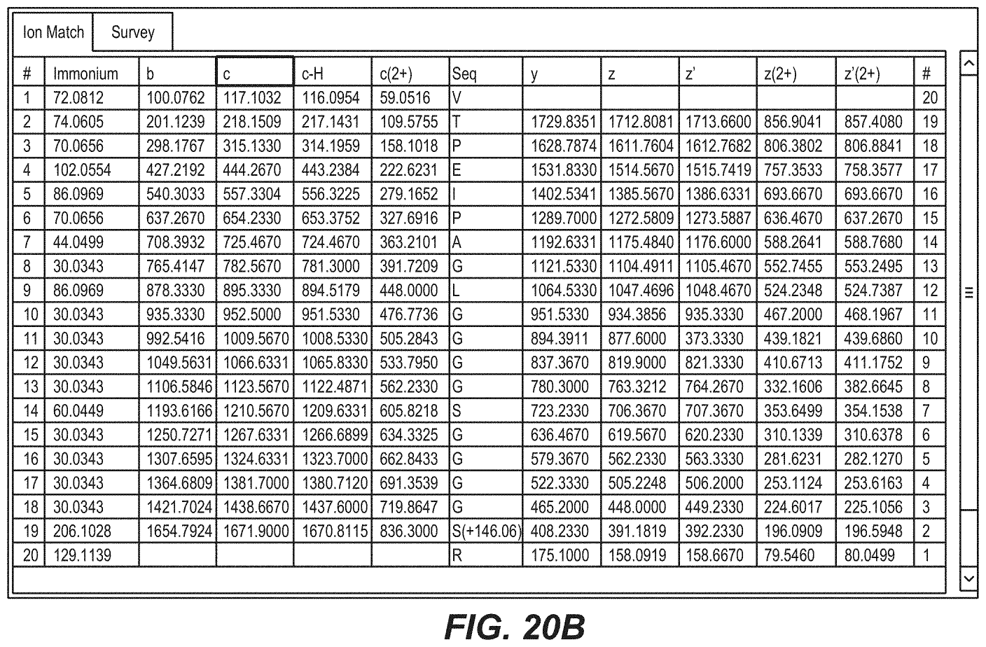

Fusion protein 4—HC4-F: with O-fucosylation of the serine residue in the linker LGGGGSGGGGSRT SEQ ID NO: 14 (cf. FIG. 18-20)

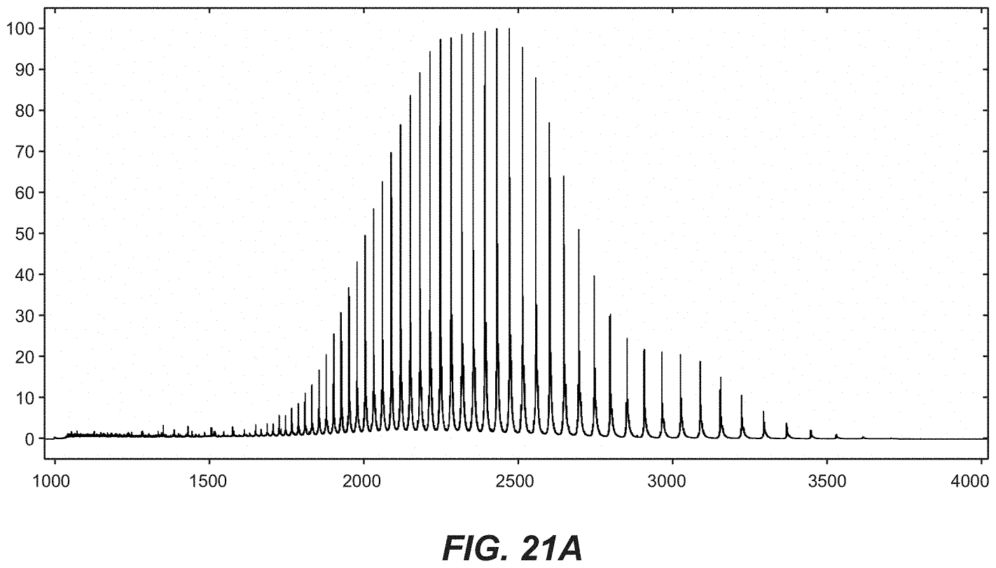

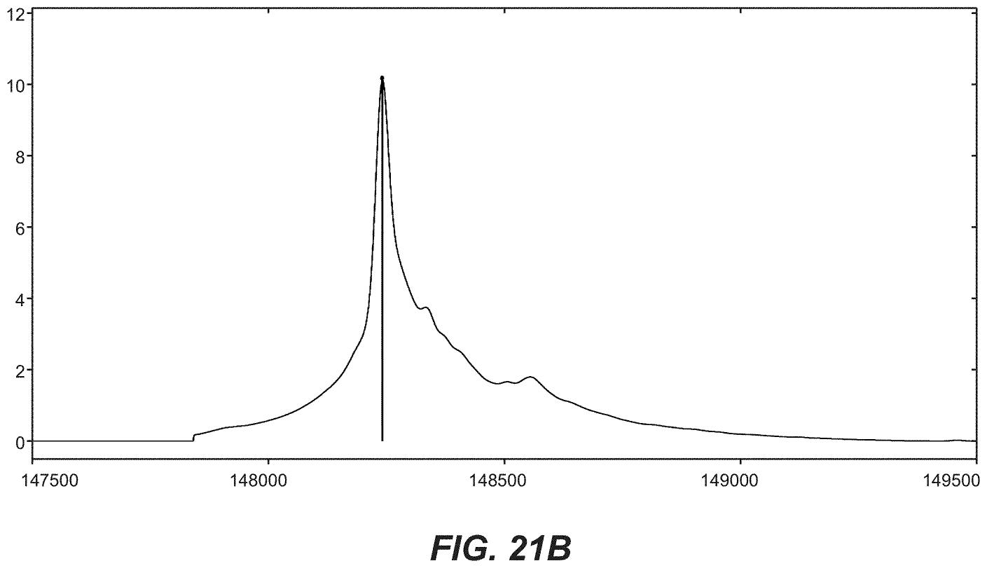

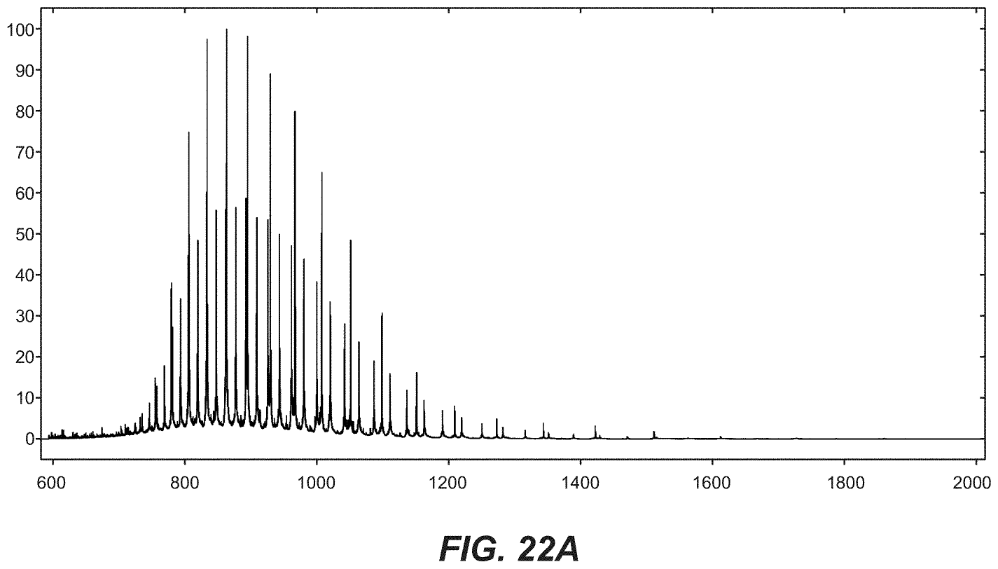

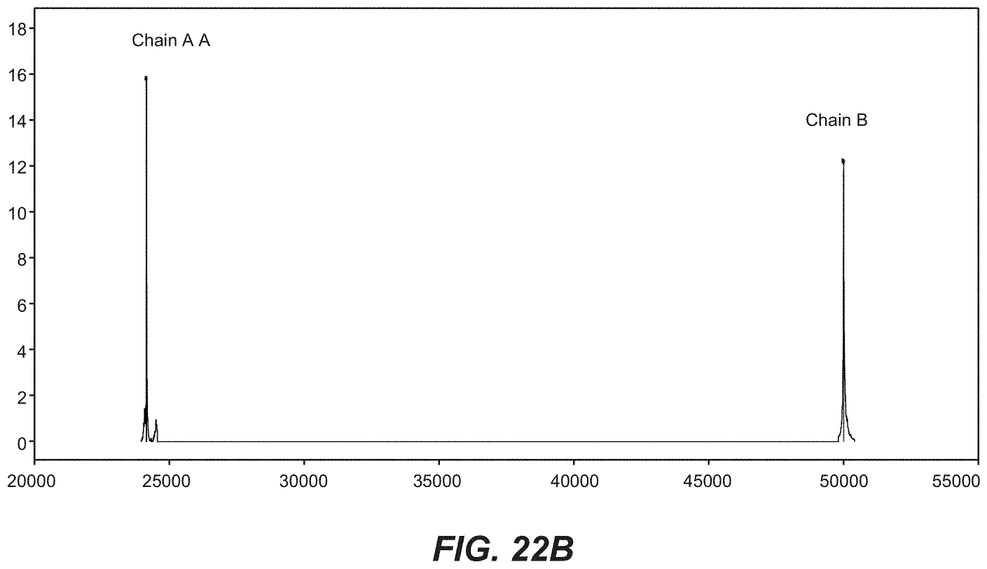

Variant of Fusion protein 4—HC4-F: without O-glycosylation in the linker comprising the amino acid sequence GGGGSGGGGSGGGGSGGGGSGGGGSGGGGGCPPC (SEQ ID NO: 23) (cf: FIG. 21-22).

Trypsin, thermolysin, neuraminidase and alkaline phosphatase from bovine intestinal mucosa were ordered from Sigma-Aldrich.

Synthetic peptides (98% HPLC-purity) were synthesized at Biosyntan GmbH.

PNGase F was obtained from Roche Diagnostics GmbH, Custom Biotech.

OpeRATOR protease was obtained from Genovis AB (Lund, Sweden; OpeRATOR is an O-protease digesting O-glycosylated proteins N-terminally of the S/T glycosylation site; the presence of O-glycans is required for OpeRATOR to exert its enzymatic activity).

### Example 1

### Enzymatic Digests

### UHR-ESI-QTOF-MS

The fusion proteins were deglycosylated using PNGase F (in presence or absence of neuraminidase), reduced in 100 mM TCEP and desalted by HPLC on a Sephadex G25 5×250 mm column (Amersham Biosciences) using 40% acetonitrile with 2% formic acid (v/v) as mobile phase. The total mass was determined by ESI-QTOF MS on a maXis 4G UHR-QTOF MS system (Bruker Daltonik) equipped with a TriVersa NanoMate source (Advion). Calibration was performed with the ESI-L Low Concentration Tuning Mix (Agilent Technologies). For visualization of the results, a software tool was used to transform the m/z spectra into deconvoluted mass spectra.

### OpeRATOR Digest

The fusion protein was N-deglycosylated with PNGase F and desialidated with neuraminidase, prior to reduction, denaturation and carboxymethylation and finally digested with the O-glycan specific endoprotease as described by the vendor (OpeRATOR, Genovis AB, Sweden). The digest was analyzed by UHR-ESI-QTOF-MS.

### Example 2

### UPLC-MS/MS Peptide Mapping

Fusion proteins were denatured and reduced in 0.3 M Tris-HCl, pH 8, comprising 6 M guanidine-HC1 and 20 mM dithiothreitol (DTT) at 37° C. for 1 hour. Thereafter the fusion proteins were alkylated by adding 40 mM iodoacetic acid (C13: 99%) (Sigma-Aldrich) and incubating at room temperature in the dark for 15 min. Excess iodoacetic acid was inactivated by adding further 20 mM DTT to the reaction mixture. The alkylated fusion protein was buffer exchanged using NAPS gel filtration columns. Afterwards the fusion proteins were proteolytically digested with trypsin in 50 mM Tris-HCl, pH 7.5, at 37° C. for 16 hours (with or without OpeRATOR protease). The reaction was stopped by adding formic acid to 0.4% (v/v). Digestions with thermolysin were performed in 25 mM Tris-HCl, 1 mM CaCl₂, pH 8.3, at 25° C. for 30 minutes and stopped by adding EDTA to 8 mM. The digested samples were stored at −80° C. and analyzed by UPLC-MS/MS using a NanoAcquity UPLC (Waters) coupled to a TriVersa NanoMate (Advion) and an Orbitrap Elite mass spectrometer (Thermo Fisher Scientific). About 2.4 μg digested fusion protein was injected in 5 μL volume. Chromatographic separation was performed by reversed-phase on a Acquity BEH300 C18 column, 1×150 mm, 1.7 μm, 300 Å (Waters) using a flow rate of 60 μL/min. The mobile phase A and B contained 0.1% (v/v) formic acid in UPLC grade water and acetonitrile, respectively. A column temperature of 50° C. was used and a gradient of 1% to 40% mobile phase B over 90 min. followed by an increase to 99% mobile phase B for 2 min. and a re-equilibration step at 1% mobile phase B for 6 min was applied. Two injections of mobile phase A were performed between sample injections using a 50 min. gradient to prevent carry-over between samples. The effluent was split post column using the TriVersa NanoMate, and a nanoliter flow portion directed into the mass spectrometer.

High-resolution MS spectra were acquired with the Orbitrap mass analyzer, and parallel detection of CID MS/MS fragment ion spectra in the ion trap with dynamic exclusion enabled (repeat count of 1, exclusion duration of 15 s (±10 ppm)). The Orbitrap Fusion was used in the data-dependent mode.

MS settings were: full MS (AGC: 2×10⁵, resolution: 6×10⁴, m/z range: 300-2000, maximum injection time: 100 ms); MS/MS (AGC: 1×10⁴, maximum injection time: 100 ms, isolation width: 2 Da). Normalized collision energy was set to 35%, activation p: 0.25, isolation width: 2 Da.

For methods where exclusively HCD MS/MS spectra were acquired, an Orbitrap full MS scan was followed by up to 20 HCD Orbitrap MS/MS spectra on the most abundant ions. The AGC for MS/MS experiments was set to 5×10⁴ at a maximum injection time of 500 ms. Normalized collision energy was set to 20%, and HCD fragmentation ions were detected in the Orbitrap at a resolution setting of 15×10³. All other settings were as described for the method using exclusively CID fragmentation.

The complementary EThcD method based on HCD and ETD as data dependent fragmentation techniques involved full scan MS acquired with the Orbitrap mass analyzer, and parallel detection of ETD and HCD fragment ion spectra in the ion trap and Orbitrap mass analyzer, respectively. A fixed cycle time was set for the full scan with as many as possible data dependent MS/MS scans.

Full MS: same setting as for CID and HCD.

For HCD, the MS/MS setting were the same as listed above.

For ETD, MS/MS the settings were as follows: reaction time was set to 50 ms, ETD reagent target: 1×10⁶, maximum injection time: 200 ms. ETD supplemental activation was enabled. Supplemental activation collision energy was set to 25%. The AGC target was set to 1×10⁴, the precursor isolation width was 2 Da and the maximum injection time was set to 250 ms.

The analysis of the LC-MS/MS data and the post-translational modification (PTM) identification was performed using the PEAKS studio 6.0 and 7.5 software (Bioinformatics Solutions Inc.) using the preprocessing option and PepFinder software (Thermo Fisher Scientific). Manual data interpretation and quantification was performed using XCalibur software (Thermo Fisher Scientific). GPMAW (Lighthouse data) was used to calculate theoretical masses and the XICs were generated with the most intense isotope mass using a mass tolerance of 8 ppm.

### Example 3

### Spiking of a Synthetic Peptides

Based on a calculated amount of the modified peptides, certain amounts of synthetic peptide were spiked into the tryptic digest. 2.4 μg tryptic digest with or without spiked synthetic peptide was analyzed by LC-MS/MS as described before.

### Example 4

### Enzymatic Dephosphorylation

Enzymatic dephosphorylation of the +79.97 Da modified tryptic linker peptide was performed by freeze drying ˜62 μg tryptic digest. The peptides were resuspended in 25 μL 100 mM Tris-HCl, 5 mM MnCl₂, pH 8.0, and incubated with 250 units of alkaline phosphatase at 37° C. for 1 h. The digested samples were stored at −80° C. MS analysis was done as described before.

It will be readily understood that the embodiments, as generally described herein, are exemplary. The presented detailed description of some embodiments as well as the presented examples are not intended to limit the scope of the present disclosure, but is merely representative of various embodiments. Moreover, the order of steps or actions of the methods disclosed herein may be changed by those skilled in the art without departing from the scope of the present disclosure. In other words, unless a specific order of steps or actions is required for proper operation of the embodiment, the order or use of specific steps or actions may be modified.

1. A linear fusion polypeptide comprising a first polypeptide domain, a peptidic linker and a second polypeptide domain, wherein
the C-terminus of the first polypeptide domain is conjugated to the N-terminus of the peptidic linker via a peptide bond and the C-terminus of the peptidic linker is conjugated to the N-terminus of the second polypeptide domain via a peptide bond,
the peptidic linker is a Gly/Ser-peptidic linker that lacks the C-terminal serine amino acid residue,
the second polypeptide domain comprises at its N-terminus any amino acid residue except for serine, threonine or proline, and
the first or the second polypeptide domain is an antibody Fc-region polypeptide, and the respective other polypeptide is selected from the group consisting of a VH domain, a VL domain, a scFv, a scFab, a VH-CH1 pair, a VL-CL pair, a VH-CL pair, a VL-CH1 pair, a receptor or extracellular domain thereof, a receptor binding portion of a ligand, an enzyme, a growth factor, an interleukin, a cytokine, or a chemokine.

2. The linear fusion polypeptide according to claim 1, wherein the linear fusion polypeptide comprises the amino acid sequence
(SEQ ID NO: 02)
GnSGmX1
wherein GnSGm is the peptidic linker with n=3 or 4 and m=3 or 4,
wherein X1 is the N-terminal amino acid residue of the second polypeptide.

3. The linear fusion polypeptide according to any one of claims 1 to 2, wherein the linear fusion polypeptide has reduced O-glycosylation compared to a fusion polypeptide wherein the second polypeptide domain has a serine or threonine or proline at its N-terminus.

4. A multimeric molecule comprising at least two polypeptides whereof at least one is a linear fusion polypeptide according to any one of claims 1 to 3.

5. The multimeric molecule according to claim 4, wherein the multimeric molecule is an antibody.

6. A nucleic acid molecule encoding a fusion polypeptide according to any one of claims 1 to 3.

7. A eukaryotic cell comprising the nucleic acid molecule according to claim 6.

8. A method of producing a linear fusion polypeptide having reduced levels of O-glycosylation comprising:
cultivating the host cell according to claim 7 under conditions where the fusion polypeptide is expressed, and
recovering the fusion polypeptide from the eukaryotic cell or the cultivation medium,
thereby producing a fusion polypeptide having reduced levels of O-glycosylation.


SEQUENCE LISTING (29 sequences)

SEQ ID NO: 1 - protein, 11 aa, Artificial Sequence
  Description of Artificial Sequence Synthetic peptide
  SITE (1)..(4): This region may encompass 1-4 residues
  SITE (6)..(10): This region may encompass 3-5 residues
  MOD_RES (11)..(11): Any amino acid except Ser, Thr, or Pro
GGGGSGGGGGX

SEQ ID NO: 2 - protein, 13 aa, Artificial Sequence
  Description of Artificial Sequence Synthetic peptide
  SITE (1)..(4): This region may encompass 1-4 residues
  SITE (6)..(10): This region may encompass 3-5 residues
  MOD_RES (11)..(11): Any amino acid except Ser, Thr, or Pro
  MOD_RES (12)..(13): Any amino acid
GGGGSGGGGGXXX

SEQ ID NO: 3 - protein, 18 aa, Artificial Sequence
  Description of Artificial Sequence Synthetic peptide
  SITE (1)..(4): This region may encompass 3-4 residues
  SITE (6)..(9): This region may encompass 3-4 residues
  SITE (11)..(15): This region may encompass 3-5 residues
  MOD_RES (16)..(16): Any amino acid except Ser, Thr, or Pro
  MOD_RES (17)..(18): Any amino acid
GGGGSGGGGSGGGGGXXX

SEQ ID NO: 4 - protein, 26 aa, Artificial Sequence
  Description of Artificial Sequence Synthetic peptide
  SITE (1)..(25): This region may encompass 3-25 residues
  MOD_RES (26)..(26): Any amino acid except Ser, Thr, or Pro
GGGGGGGGGGGGGGGGGGGGGGGGGX

SEQ ID NO: 5 - protein, 28 aa, Artificial Sequence
  Description of Artificial Sequence Synthetic peptide
  SITE (1)..(25): This region may encompass 3-25 residues
  MOD_RES (26)..(26): Any amino acid except Ser, Thr, or Pro
  MOD_RES (27)..(28): Any amino acid
GGGGGGGGGGGGGGGGGGGGGGGGGXXX

SEQ ID NO: 6 - protein, 13 aa, Artificial Sequence
  Description of Artificial Sequence Synthetic peptide
  SITE (1)..(4): This region may encompass 1-4 residues
  SITE (6)..(10): This region may encompass 3-5 residues
  MOD_RES (11)..(11): Ser, Thr, or Pro
  MOD_RES (12)..(13): Any amino acid
GGGGSGGGGGXXX

SEQ ID NO: 7 - protein, 11 aa, Artificial Sequence
  Description of Artificial Sequence Synthetic peptide
  A polypeptide fragment is attached at the N-terminal end of the sequence
GGGGSGGGGSR

SEQ ID NO: 8 - protein, 11 aa, Artificial Sequence
  Description of Artificial Sequence Synthetic peptide
  A polypeptide fragment is attached at the N-terminal end of the sequence
  MOD_RES (10)..(10): Phosphorylated Ser
GGGGSGGGGSR

SEQ ID NO: 9 - protein, 12 aa, Artificial Sequence
  Description of Artificial Sequence Synthetic peptide
  A polypeptide fragment is attached at the N- and C-terminal ends of the sequence
  MOD_RES (10)..(10): Phosphorylated Ser
GGGGSGGGGSRE

SEQ ID NO: 10 - protein, 28 aa, Artificial Sequence
  Description of Artificial Sequence Synthetic peptide
  MOD_RES (25)..(25): Glycosylated Ser
SLSLPGGGGSGGGGSGGGGSGGGGSIQM

SEQ ID NO: 11 - protein, 47 aa, Artificial Sequence
  Description of Artificial Sequence Synthetic polypeptide
  LC3-F fragment
  MOD_RES (24)..(24): Glycosylated Thr
GGGSGGGGSGGGGSGGGGSGGGGTCPPCPAPEAAGGPSVFLFPPKPK

SEQ ID NO: 12 - protein, 23 aa, Artificial Sequence
  Description of Artificial Sequence Synthetic peptide
  LC3-F tryptic digest with OpeRATOR protease
TCPPCPAPEAAGGPSVFLFPPKP

SEQ ID NO: 13 - protein, 49 aa, Artificial Sequence
  Description of Artificial Sequence Synthetic polypeptide
  LC3-F tryptic digest without OpeRATOR protease
  MOD_RES (1)..(2): Any naturally occurring amino acid
XXGGGSGGGGSGGGGSGGGGSGGGGTCPPCPAPEAAGGPSVFLFPPKPK

SEQ ID NO: 14 - protein, 13 aa, Artificial Sequence
  Description of Artificial Sequence Synthetic peptide
  HC4-F fragment
  MOD_RES (11)..(11): Fucosylated Ser
LGGGGSGGGGSRT

SEQ ID NO: 15 - protein, 13 aa, Artificial Sequence
  Description of Artificial Sequence Synthetic peptide
  C-terminal amino acid residue of domain 1 is attached at the N-terminal end of the sequence
  MOD_RES (11)..(11): Fucosylated Ser
LGGGGSGGGGSRT

SEQ ID NO: 16 - protein, 4 aa, Artificial Sequence
  Description of Artificial Sequence Synthetic peptide
GGGS

SEQ ID NO: 17 - protein, 5 aa, Artificial Sequence
  Description of Artificial Sequence Synthetic peptide
GGGGS

SEQ ID NO: 18 - protein, 5 aa, Artificial Sequence
  Description of Artificial Sequence Synthetic peptide
GGGGP

SEQ ID NO: 19 - protein, 12 aa, Artificial Sequence
  Description of Artificial Sequence Synthetic peptide
GGGGSGGGGSRE

SEQ ID NO: 20 - protein, 12 aa, Artificial Sequence
  Description of Artificial Sequence Synthetic peptide
GGGGSGGGGSRT

SEQ ID NO: 21 - protein, 8 aa, Artificial Sequence
  Description of Artificial Sequence Synthetic peptide
GGGSGGGS

SEQ ID NO: 22 - protein, 10 aa, Artificial Sequence
  Description of Artificial Sequence Synthetic peptide
GGGGSGGGGS

SEQ ID NO: 23 - protein, 34 aa, Artificial Sequence
  Description of Artificial Sequence Synthetic polypeptide
GGGGSGGGGSGGGGSGGGGSGGGGSGGGGGCPPC

SEQ ID NO: 24 - protein, 13 aa, Artificial Sequence
  Description of Artificial Sequence Synthetic peptide
  A polypeptide fragment is attached at the N-terminal end of the sequence
LGGGGSGGGGSRT

SEQ ID NO: 25 - protein, 10 aa, Artificial Sequence
  Description of Artificial Sequence Synthetic peptide
  SITE (1)..(4): This region may encompass 3-4 residues
  SITE (6)..(9): This region may encompass 3-4 residues
  MOD_RES (10)..(10): Any amino acid except Ser, Thr, or Pro
GGGGSGGGGX

SEQ ID NO: 26 - protein, 18 aa, Artificial Sequence
  Description of Artificial Sequence Synthetic peptide
  SITE (1)..(4): This region may encompass 1-4 residues
  SITE (6)..(9): This region may encompass 1-4 residues
  SITE (11)..(15): This region may encompass 3-5 residues
  MOD_RES (16)..(16): Ser, Thr, or Pro
  MOD_RES (17)..(18): Any amino acid
GGGGSGGGGSGGGGGXXX

SEQ ID NO: 27 - protein, 18 aa, Artificial Sequence
  Description of Artificial Sequence Synthetic peptide
  SITE (1)..(4): This region may encompass 3-4 residues
  SITE (6)..(9): This region may encompass 1-4 residues
  SITE (11)..(15): This region may encompass 3-5 residues
  MOD_RES (16)..(16): Any amino acid except Ser, Thr, or Pro
  MOD_RES (17)..(18): Any amino acid
GGGGSGGGGSGGGGGXXX

SEQ ID NO: 28 - protein, 31 aa, Artificial Sequence
  Description of Artificial Sequence Synthetic polypeptide
SLSLSPGGGGGSGGGGSGGGGSGGGGSIQMT

SEQ ID NO: 29 - protein, 20 aa, Artificial Sequence
  Description of Artificial Sequence Synthetic peptide
VTPEIPAGLGGGGSGGGGSR